(19) United States
(12) Patent Application Publication
(10) Pub. No.: US 2025/0014673 A1
(43) Pub. Date: Jan. 9, 2025

(54) SYSTEMS AND METHODS FOR POLYMER SIDE-CHAIN CONFORMATION PREDICTION

(71) Applicant: ZYMEWORKS BC INC., Vancouver (CA)

(72) Inventors: Abhishek MUKHOPADHYAY, VANCOUVER (CA); Surjit Bhimarao Dixit, Vancouver (CA); Alejandro Gil Ley, Vancouver (CA); James Liam McWhirter, Vancouver (CA); Benjamin John McMaster, Vancouver (CA); Amit Kadan, Vancouver (CA)

(21) Appl. No.: 18/705,894

(22) PCT Filed: Nov. 1, 2022
(86) PCT No.: PCT/CA2022/051612

§ 371 Date: Apr. 29, 2024

Related U.S. Application Data
(60) Provisional application No. 63/274,444, filed on Nov. 1, 2021.

(51) Int. Cl. G16B 15/00; G16B 40/00

(52) U.S. Cl. CPC G16B 15/00; G16B 40/00

(57) ABSTRACT

Disclosed systems and methods obtain a graph of a polymer comprising nodes and edges, the nodes representing polymer atoms, the edges encoding distances and relative orientations between corresponding pairs of nodes and whether pairs of nodes are covalently bound. Each subgraph in a plurality of first partial-context subgraphs of the graph is sequentially inputted into a first model to calculate first side chain dihedral angles for polymer residues. This updates the graph through first side chain dihedral angles. Each second subgraph in a plurality of second partial-context subgraphs of the updated graph is inputted into a second model, thereby obtaining calculated second side chain dihedral angles for polymer residues that serve to update the graph through second side chain dihedral angles. The graph is again updated with updated side chain dihedral angle values obtained by sequentially inputting full-context subgraphs, each such subgraph representing a different residue, into a full-context model.

### CROSS REFERENCE TO RELATED APPLICATIONS

This application claims priority to U.S. Provisional Patent Application No. 63/274,444 entitled “SYSTEMS AND METHODS FOR POLYMER SIDE-CHAIN CONFORMATION PREDICTION,” filed Nov. 1, 2022, which is hereby incorporated by reference.

### TECHNICAL FIELD

The disclosed embodiments relate generally to systems and methods for molecular modelling of polymers.

### BACKGROUND

Accurate prediction of side-chain conformation is an important component in protein modeling, mutagenesis, protein structure prediction and protein engineering and design problems. Side-chains geometry is also key for recognition of binding site, for in-silico binding affinity assessment and for interface engineering between cognate binding proteins and protein/ligand complexes.

For structure refinement methods that include backbone conformation change, one stage in the refinement process is prediction of side-chain conformation, also known as repacking. While accuracy is important, speed is key for refining a large ensemble of decoys with different backbone geometry using in-silico methods.

Conventional methods that attempt to solve the protein side-chain packing problem comprise broadly of three components (i) a discrete rotamer library, (ii) an energy/scoring function and (iii) a search algorithm. Such methods select a set of rotamers (one rotamer for each amino acid) from the rotamer library to minimize the given energy function. Such conventional protein side-chain packing methods typically use search algorithms to predict a set of rotamers from a rotamer library for the region of interest in a protein that minimizes the energy/scoring function. A rotamer library is a collection of frequencies, mean dihedral angles, and standard deviations of the discrete conformations (rotamers) of the amino acid side chains derived from proteins in the crystal PDB database. See Dunbrack, 2002, “Rotamer libraries in the 21st century,” Current Opinion in Structural Biology 12(4), pp. 431-40; Xiang and Honig, 2001, “Extending the Accuracy Limits of Prediction for Side-chain Conformations,” J. Mol. Biol. 31, pp. 421-430; Shapovalov and Dunbrack, 2011, “A smoothed backbone-dependent rotamer library for proteins derived from adaptive kernel density estimates and regressions,” Structure 19(6), pp. 844-858; Dunbrack and Karplus, 1993, “Backbone-dependent rotamer library for proteins, Application to side chain prediction,” J. Mol. Biol. 230: 543-574; Lovell et al., 2000, “The Penultimate Rotamer Library,” Proteins: Structure Function and Genetics 40: 389-408, and Xiang, 2001, “Extending the Accuracy Limits of Prediction for Side-chain Conformations,” Journal of Molecular Biology 311, p. 421, each of which is hereby incorporated herein by reference. Two broad categories of rotamer libraries include (i) backbone-dependent rotamer libraries (BBDRL), where the frequencies, mean dihedral angles, and standard deviations of the rotamers are a function of the protein backbone dihedral angles and (ii) backbone-independent rotamer libraries (BBIRL) where the frequencies and mean dihedral angles are independent of the backbone conformation. The performance of BBDRL and BBIRL methods relies heavily upon the richness and quality of the rotamer library, accuracy of the energy functions or the rigour of the sampling techniques.

Conventional sidechain packing algorithms include Krivov et al., 2009, “Improved prediction of protein side-chain conformations with SCWRL4,” Proteins: Structure, Function, and Bioinformatics 77(4), pp. 778-795; Miao et al., 2011, “RASP: rapid modeling of protein side chain conformations,” Bioinformatics 27(22), pp. 3117-3122; Cao et al., 2011, “Improved side-chain modeling by coupling clash-detection guided iterative search with rotamer relaxation,” Bioinformatics 27(6), pp. 785-790; Nagata et al., 2012, “SIDEpro: A novel machine learning approach for the fast and accurate prediction of side-chain conformations,” Proteins: Structure, Function, and Bioinformatics 80(1), pp. 142-153; Huang et al., 2020, “FASPR: an open-source tool for fast and accurate protein side-chain packing,” Bioinformatics 36(12), pp. 3758-3765; Liu et al., Prediction of amino acid side chain conformation using a deep neural network, arXiv preprint arXiv:1707.08381 (2017); International Patent Publication No. WO2017196963A1; and Xu et al., 2020 “Opus-rota3: Improving protein side-chain modeling by deep neural networks and ensemble methods,” Journal of Chemical Information and Modeling 60(12), pp. 6691-6697, each of which is hereby incorporated herein by reference.

One such conventional side-chain packing method, SCWRL4, Krivov et al., 2009, “Improved prediction of protein side-chain conformations with SCWRL4,” Proteins: Structure, Function, and Bioinformatics 77(4), pp. 778-795, considers two distinct types of models, namely a rigid rotamer model (RRM) and a flexible rotamer model (FRM). In the RRM approach, a single-body term scores a rotamer relative to the most abundant rotamer given the backbone dihedrals, in addition to a score pertaining to a side chain interaction term with the backbone, ligand or other fixed atoms in the system. These pairwise terms consist of tuned repulsive and attractive Van der Waals interactions and hydrogen bonding. In the FRM approach, subrotamers, e.g., conformations that differ in one or more dihedral angles by one standard deviation from the mean values given in the rotamer library are also considered. SCWRL4 uses a deterministic search method, where the inter-residue interactions are represented as a graph and the combinatorial optimization is performed via edge decomposition, application of the dead-end elimination (DEE) algorithm and tree decomposition. SCWRL4 includes a feature that allows consideration of the crystal symmetry in the side-chain conformation prediction.

Another such conventional side-chain packing method, OPUS-Rota, comprises two stages (i) a sidechain rotamer prediction method based on deep neural networks, named OPUSRotaNN, and (ii) a side-chain modeling framework, named OPUS-Rota3, which integrate the results of different methods to predict rotamers along with the SCRWL4 BBDRL to form an ensemble method. See, Xu et al., 2020 “Opus-rota3: Improving protein side-chain modeling by deep neural networks and ensemble methods,” Journal of Chemical Information and Modeling 60(12), pp. 6691-6697. For OPUSRotaNN, a deep learning model was trained using 241 input features that includes position-specific scoring matrix (PSSM) features, hidden Markov model (HHM) features, physicochemical properties, proteomics signature profiling (PSP) features, protein backbone torsion angles, 3- and 8-state secondary structure (SS) features and contact environment information. The training model comprises a convolutional neural network (CNN) component, a bidirectional long-short-term memory (LSTM) component, and a modified transformer component. The output of the neural network are the sine and cosine of all side chain dihedral angles (where available). The predicted side chains dihedral angles are then included in BBDRL for the final stage of the ensemble-based side chain modeling program, OPUS-Rota3. The output candidates from other methods, including OPUSRotaNN, were reweighted and included in BBDRL to perform sampling using their custom scoring function comprised of a side chain conformation-based energy term, Van der Waals like pair energy terms and a rotamer-frequency based energy term.

Other methods that have successfully attempted to solve the side chain prediction problem to varied degree of accuracy or computational efficiency are RASP (See, Miao et al., 2011, “RASP: rapid modeling of protein side chain conformations,” Bioinformatics 27(22), pp. 3117-3122), CISRR (See, Cao et al., 2011, “Improved side-chain modeling by coupling clash-detection guided iterative search with rotamer relaxation,” Bioinformatics 27(6), pp. 785-790), SIDEpro [See, Nagata et al., 2012, “SIDEpro: A novel machine learning approach for the fast and accurate prediction of side-chain conformations,” Proteins: Structure, Function, and Bioinformatics 80(1), pp. 142-153], and FASPR (See, Huang et al., 2020, “FASPR: an open-source tool for fast and accurate protein side-chain packing,” Bioinformatics 36(12), pp. 3758-3765). Each of these methods use some combination and variation of the energy functions and the rotamer search algorithms found in OPUS-Rota and SCWRL4 that are described above.

While the backbone conformation change may be minimal and structure prediction can often be accomplished by accurately predicting the side-chain conformation of the mutated region only in single-site mutants and in homologous proteins, application of the above-identified algorithms to more complex protein-packing problems such as determining rotamers of each residue in an entire protein or using starting models other than homologous proteins, have drawbacks.

One such drawback of these sampling-based approaches is that both accuracy and computational efficiency of these methods rely on the quality and type of rotamer library used to sample from and involves formulating fine-tuned, heavily approximated scoring terms which are not necessarily derived using first principles. For instance, in BBDRL, rotamer statistics depend upon the backbone dihedral angles, which as intended narrows the search space but that can often miss the true rotamer if that rotamer observes low frequency in PDB. Rotamer libraries, including the ones that depend upon backbone torsion angles, are too generic, which results in excessive computational burden being placed on sifting through rotamers that are highly unlikely for the local environment of the residue in question; an ideal rotamer library should also be able to capture higher order dependence on the local environment. The scoring functions, on the other hand, due to analytical complexity and heavy computational costs, do not account for interactions like electrostatics or solvation energy. Furthermore, energy terms like non-covalent interactions energies involving aromatic π−π stacking is poorly captured via tractable analytical or empirical models.

Given the above background, there is a clear need for improved systems and methods for side chain packing.

### SUMMARY

The present disclosure addresses the need in the art. Disclosed are systems and methods for determining the side-chain rotamers for all or a substantial portion of the residues in a polymer, such as a protein, without reliance on computationally intensive energy functions or extensive side chain rotamer libraries. This is done with a computational framework that is based on a geometric learning approach that allows one to predict polymer side chain conformations directly without need for a rotamer library, a scoring function, or a sampling algorithm. In the proposed computational framework, the protein structures are represented as graphs where the sequence and structural details are embedded into the node and the edge attributes. A set of models, each with a different degree of structural detail and for protein side chain prediction, are trained. These trained models are applied sequentially and iteratively, starting from only the protein backbone description as disclosed herein. This iteration is done first through a series of partial-context models and second through a series of full-context models to build out the side chain rotamer angles of the polymer. In some embodiments, each of the partial-context and full-context models is a graph neural network. In graph neural networks, the representation vector of nodes and edges is computed and updated by recursively aggregating and transforming node-edge representation vectors of its neighbors defined via an adjacency matrix.

In more detail, a computer system for molecular modeling is provided. The computer system comprises one or more processors and memory addressable by the one or more processors. The memory stores at least one program for execution by the one or more processors.

The at least one program comprises instructions for (A) obtaining a graph of at least a portion of a polymer. The graph comprises a plurality of nodes and a plurality of edges. For example, in some embodiments, each node in the plurality of nodes represents an atom as a tuple that includes an encoding of residue type of the residue the atom is in and an encoding of the name of the atom in the residue. In some embodiments, a node attribute is the tuple of residue name and atom type that is fed as categorical variables using a set of integers between 1 and N, where N is the total number of distinct residues name—atom type combinations. In such embodiments, these integers are inputted into an embedding layer.

Initially, each node in the plurality of nodes represents a main chain atom of the polymer. At later stages nodes are added to the plurality of nodes for atoms of side chains of the polymer. Each respective edge in the plurality of edges encodes at least (i) a corresponding distance relationship (e.g., relative orientation in three-dimensional space) between a corresponding pair of nodes in the plurality of nodes and (ii) a binary indicator that indicates whether or not the corresponding pair of nodes represents a pair of atoms covalently bound to each other in the polymer. The referenced portion of the polymer comprises a plurality of residues, at least two of which have one or more side chain dihedral angles in a set of side chain dihedral angles. In typical embodiments, the graph initially represents all the backbone atoms of each residue of the polymer.

In typical embodiments, each residue in the plurality of residues is one of twenty naturally occurring amino acids. In typical embodiments, the polymer is a polypeptide. In one example embodiment, the polymer is an antigen-antibody complex.

To give a sense of scale, in some embodiments, the plurality of residues represented by the graph comprises 50 or more residues of the polymer.

In some embodiments, the plurality of residues comprises each residue of the polymer.

In some embodiments, the polymer represents a single crystal asymmetric unit. In other embodiments, the plurality of residues includes one or more second residues that are crystallographic symmetry mates of one or more first residues in the plurality of residues and the graph includes a definition of the default asymmetric unit of the polymer.

In some embodiments, the corresponding distance relationship between a corresponding pair of nodes i and j in the plurality of nodes represented by an edge is of the form e^{-r}^{ᵢⱼ}^{²}^{/k}, where r is a distance between three-dimensional coordinates for node i and three-dimensional coordinates for node j, and κ is the square of a cuttoff distance. In some such embodiments, rᵢⱼ is in units of Å and κ is 100 Å².

In some embodiments, each respective edge in the plurality of edges further encodes a directional feature between a corresponding pair of nodes. For example, in some such embodiments, for each of pair of atoms, i and j in the polymer, there are two different edges, eᵢⱼ which is from i to j, and eⱼᵢ, which is from j to i. In such embodiments, both eᵢⱼ and eⱼᵢ include five features, two of which are the same. The first feature is distance (a corresponding distance relationship between i and j), as discussed above, which is the same for eᵢⱼ and eⱼᵢ. The second feature is a binary indicator that indicates whether or not i and j are covalently bound to each other in the polymer, as discussed above, which is the same for eᵢⱼ and eⱼᵢ. The remaining three features eᵢⱼ and eⱼᵢ encode the directional vector from atom i to atom j, in the case of eⱼᵢ and the directional vector from atom j to atom i, in the case of eⱼᵢ. Thus, there is a representation of 1×5 for each direction in the bidirectional graph. The directional vector (encoded as the final three features) for eᵢⱼ and eⱼᵢ is specific to the direction based upon the local coordinate system placed on i and similarly on j.

In some embodiments, given a protein backbone, the side chain C_{β} atom in each target residue side chain is then deterministically predicted using a conventional C_{β} residue builder tool based on the coordinates of the backbone atoms of the polymer in the set of M three-dimensional coordinates {x₁, . . . , x_{M}}. Once the coordinates of the C_{β} atom for the target residue are populated, they are included in the graph 102. That is, a node is added to the graph for each C_{β} atom and edges between such C_{β} atoms and other atoms in the graph are added.

The at least one program further comprises instructions for (B) sequentially inputting each first partial-context subgraph in a plurality of first partial-context subgraphs of the graph into a first trained partial-context graph neural network thereby obtaining a plurality of first instances of calculated first side chain dihedral angles for the plurality of residues. In typical embodiments the first trained partial-context graph neural network has numerous parameters, for instance at least 500 parameters, that have been refined through training against test data prior to inputting each first partial-context subgraph into the model. In more detail, in some embodiments, the sequentially inputting (B) comprises, for each respective residue in the plurality of residues, inputting a corresponding first partial-context subgraph, in the plurality of first partial-context subgraphs of the graph, drawn from the nodes in the graph that represent backbone atoms and the C_{β} atom of the respective residue or backbone atoms and the C_{β} atoms of the polymer proximate to the respective residue, into the first trained partial-context graph neural network, thereby obtaining a first instance of a corresponding calculated first side chain dihedral angle for the respective residue. In some such embodiments, the backbone atoms of the polymer proximate to the respective residue are a cutoff number of atoms (e.g., between 20 and 80 atoms) in the protein that are closest to the respective residue. In some such embodiments, the first instance of the corresponding calculated first side chain dihedral angle for the respective residue is the X₁ side chain dihedral angle for the respective residue.

The at least one program further comprises instructions for (C) updating the graph up to the first side chain dihedral angle (e.g., the X₁ side chain dihedral angle) of each residue in the plurality of residues using the plurality of first instances of calculated first side chain dihedral angles. In more detail, in some embodiments, the updating (C) comprises, for each respective residue in the plurality of residues, using the corresponding first instance of the corresponding calculated first side chain dihedral angle to update the graph of the polymer to include nodes and edges for atoms of the respective residue up to the first side chain dihedral angle of the respective residue.

The at least one program further comprises instructions for (D) sequentially inputting each second partial-context subgraph in a plurality of second partial-context subgraphs of the graph into a second trained partial-context graph neural network thereby obtaining a plurality of first instances of calculated second side chain dihedral angles for residues in the plurality of residues. In typical embodiments the second trained partial-context graph neural network has numerous parameters, for instance at least 500 parameters, that have been refined through training against test data prior to inputting each first partial-context subgraph into the model. In more detail, in some embodiments, the sequentially inputting (D) comprises, for each respective residue in the plurality of residues having a second side chain dihedral angle, inputting a corresponding second partial-context subgraph, in the plurality of second partial-context subgraphs of the graph, drawn from the nodes in the graph that represent backbone atoms or side chain atoms of up to the first side chain dihedral angle of (i) the respective residue or (ii) residues proximate to the respective residue, into the second trained partial-context graph neural network, thereby obtaining a first instance of a corresponding calculated second side chain dihedral angle for the respective residue. It will be appreciated that, in some embodiments, not all of the plurality of residues will have a second side chain dihedral angle. In some embodiments, the first instance of the corresponding calculated second side chain dihedral angle for the respective residue is the X₂ side chain dihedral angle for the respective residue.

The at least one program comprises instructions for (E) updating the graph up to the level of the second side chain dihedral angles using the plurality of first instances of calculated second side chain dihedral angles. In more detail, in some embodiments, the updating (E) comprises, for each respective residue in the plurality of residues having a second side chain dihedral angle, using the corresponding first instance of the corresponding calculated second side chain dihedral angle to update the graph to include nodes and edges for atoms of the respective residue up to the second dihedral angle.

The at least one program further comprises instructions for (F) updating the graph with updated side chain dihedral angle values obtained by sequentially inputting a plurality of full-context subgraphs, each full-context subgraph in the plurality of full-context subgraphs associated with a different residue in the plurality of residues, into a plurality of trained full-context graph neural networks thereby elucidating the side chain dihedral angle values for the plurality of residues. In typical embodiments, each trained full-context graph neural network in the plurality of trained full-context graph neural networks numerous parameters, for instance at least 500 parameters, that have been refined through training against test data prior to sequentially inputting each full-context subgraph into the respective models.

In some embodiments, the updating (F) comprises the following procedure. First, (i), for each respective residue in the plurality of residues, a corresponding first full-context subgraph drawn from the nodes in the graph representing heavy (non-hydrogen) atoms, other than side chain atoms beyond the C_{β} carbon of the respective residue, is inputted into a first trained full-context graph neural network in the plurality of trained full-context graph neural networks, thereby obtaining a second instance of a corresponding calculated first side chain dihedral angle for the respective residue. Second, (ii), for each respective residue in the plurality of residues, use the second instance of the corresponding calculated first side chain dihedral angle to update the corresponding distance relationship of edges in the graph affected by the second instance of the corresponding calculated first side chain dihedral angle. Third, (iii), for each respective residue in the plurality of residues having a second side chain dihedral angle, input a corresponding second full-context subgraph drawn from the nodes in the graph, other than side chain atoms of the respective residue beyond the first dihedral angle, into a second trained full-context graph neural network in the plurality of trained full-context graph neural networks, thereby obtaining a second instance of a corresponding calculated second side chain dihedral angle for the respective residue. Fourth, (iv), for each respective residue in the plurality of residues having a second side chain dihedral angle, use the second instance of the corresponding calculated second side chain dihedral angle to update the distance relationship of each edge in the graph affected by the second instance of the corresponding calculated second side chain dihedral angle.

In some embodiments, the first trained partial-context graph neural network, the second trained partial-context graph neural network, and each trained full-context graph neural network in the plurality of trained full-context graph neural networks is a message passing graph neural network.

In some embodiments, the first trained partial-context graph neural network, the second trained partial-context graph neural network, and each trained full-context graph neural network in the plurality of trained full-context graph neural networks comprises an embedding layer for receiving embedded graph information associated with a residue in the polymer, followed by a plurality of layers that each convolve over both a plurality of edge attributes and a plurality of node attributes, followed by an average pooling layer employed to the nodes corresponding to atoms in the respective residue, followed by a multi-layered perceptron with an activation function (e.g., tanh) having two output channels, where the output channels give a sine and a cosine value for a side chain dihedral angle of the respective residue.

In some embodiments, prior to the updating (F), for each respective residue in the plurality of residues having a X₃ dihedral angle, the at least one program further comprises instructions for inputting a corresponding third partial-context subgraph drawn from the nodes in the graph that represent backbone atoms or side chain atoms of up to the second side chain dihedral angle of (i) the respective residue or (ii) residues proximate to the respective residue, into a third trained partial-context graph neural network thereby obtaining a first instance of a corresponding calculated X₃ dihedral angle for the respective residue. In typical embodiments the third trained partial-context graph neural network has numerous parameters, for instance at least 500 parameters, that have been refined through training against test data prior to inputting each first partial-context subgraph into the model. For each respective residue in the plurality of residues having a X₃ dihedral angle, the corresponding first instance of the corresponding calculated X₃ dihedral angle is used to update the graph to include nodes and edges for atoms of the respective residue up to the X₃ dihedral angle. In such embodiments, the updating (F) further comprises (v) for each respective residue in the plurality of residues having a X₃ dihedral angle, inputting a corresponding third full-context subgraph drawn from the nodes in the graph, other than side chain atoms of the respective residue beyond the second dihedral angle, into a third trained full-context graph neural network in the plurality of trained full-context graph neural networks, thereby obtaining a second instance of a corresponding calculated X₃ dihedral angle for the respective residue, and (vi) for each respective residue in the plurality of residues having a X₃ dihedral angle, using the second instance of the corresponding calculated X₃ dihedral angle to update the distance relationship of each edge in the graph affected by the second instance of the corresponding calculated X₃ dihedral angle.

In some embodiments, prior to the updating (F), for each respective residue in the plurality of residues having a X₄ dihedral angle, the at least one program further comprises instructions for inputting a corresponding fourth partial-context subgraph drawn from the nodes in the graph that represent backbone atoms or side chain atoms of up to the X₃ dihedral angle of (i) the respective residue or (ii) residues proximate to the respective residue, into a fourth trained partial-context graph neural network thereby obtaining a first instance of a corresponding calculated X₄ dihedral angle for the respective residue. In typical embodiments, the fourth trained partial-context graph neural network has numerous parameters, for instance at least 500 parameters, that have been refined through training against test data prior to inputting each first partial-context subgraph into the model. For each respective residue in the plurality of residues having a X₄ dihedral angle, the corresponding first instance of the corresponding calculated X₄ dihedral angle is used to update the graph to include nodes and edges for atoms of the respective residue through the X₄ dihedral angle. In such embodiments, the updating (F) further comprises: (vi) for each respective residue in the plurality of residues having a X₄ dihedral angle, inputting a corresponding fourth full-context subgraph drawn from the nodes in the graph, other than side chain atoms of the respective residue beyond the X₃ angle, into a fourth trained full-context graph neural network in the plurality of trained full-context graph neural networks, thereby obtaining a second instance of a corresponding calculated X₄ dihedral angle for the respective residue, and (vi) for each respective residue in the plurality of residues having a X₄ dihedral angle, using the second instance of the corresponding calculated X₄ dihedral angle to update the distance relationship of each edge in the graph affected by the second instance of the corresponding calculated X₄ dihedral angle.

In some embodiments, the at least one program further comprises instructions for repeating the sequentially inputting (B), updating (C), sequentially inputting (D), updating (E), and updating (F) until a side chain dihedral angle convergence criterion is satisfied. In some such embodiments, the side chain dihedral angle convergence criterion is an average change in side chain dihedral angle across the plurality of residues after repetition of the sequentially inputting (B), updating (C), sequentially inputting (D), updating (E), and updating (F) dropping below a threshold value.

In some embodiments, the at least one program further comprises instructions for training the first trained partial-context graph neural network, the second trained partial-context graph neural network, and each trained full-context graph neural network in the plurality of trained full-context graph neural networks using a loss function that trains unambiguous side chain dihedral angles as a regression task and ambiguous side chain dihedral angles by considering the lower of the two possible losses attributable to the ambiguous side chain dihedral angle Xᵢ. In some embodiments, the regression task is a mean squared error loss function, a mean absolute error loss function, a Huber loss function, a Log-Cosh loss function, or a quantile loss function.

In some embodiments, a first loss in the two possible losses is for a side chain dihedral angle value for Xᵢ and the second loss in the two possible losses is a for a side chain dihedral angle value for Xᵢ−π.

In some embodiments, the at least one program further comprises instructions for using the elucidated side chain dihedral angle values for the plurality of residues to determine an interaction score between the polymer and a composition.

In some such embodiments, the polymer is an enzyme, the composition is being screened in silico to assess an ability to inhibit an activity of the enzyme, and the interaction score is a calculated binding coefficient of the composition to the first enzyme.

In some such embodiments, the polymer is a first protein, the composition is a second protein being screened in silico to assess an ability to bind to the first protein in order to inhibit or enhance an activity of the first protein, and the interaction score is a calculated binding coefficient of the second protein to the first protein.

In some such embodiments, the polymer is a first Fc fragment of a first type, the composition is a second protein is Fc fragment of a second type, and the interaction score is a calculated binding coefficient of the second Fc fragment to the first Fc fragment.

In some embodiments, the polymer is a protein with one or more mutations introduced into the protein and the at least one program further comprises instructions for using the elucidated side chain dihedral angle values for the plurality of residues to determine an effect of the one or more mutations on an activity of the protein relative to an activity of a wild-type naturally occurring version of the protein.

Another aspect of the present disclosure provides a non-transitory computer readable storage medium storing one or more computational modules for molecular modeling, the one or more computational modules collectively comprising instructions for performing any of the methods disclosed herein, including those performed by the at least one program of the computer systems disclosed herein.

### BRIEF DESCRIPTION OF THE DRAWINGS

The embodiments disclosed herein are illustrated by way of example, and not by way of limitation, in the figures of the accompanying drawings. Like reference numerals refer to corresponding parts throughout the drawings.

FIGS. 1A, 1, 1C, and 1D collectively provide a block diagram illustrating a system, according to an embodiment of the present disclosure.

FIGS. 2A, 2B, 2C, 2D, 2E, 2F, 2G, 2H, 2I, and 2K illustrate method for molecular modeling according to various embodiments of the present disclosure, where optional elements are indicated by dashed boxes.

FIG. 3 illustrates the encoding of a protein crystal structure into a graph representation with nodes representing atoms and geometric relations with neighboring atoms represented by edges in accordance with an embodiment of the present disclosure.

FIG. 4 illustrates information that is encoded into edges of a graph to provide the structural and topological information of a polymer in a translationally and rotationally invariant manner in accordance with an embodiment of the present disclosure.

FIG. 5 illustrates an overview of the input into a model for determining a target side chain dihedral angle in a target residue of a target protein in accordance with an embodiment of the present disclosure.

FIG. 6 illustrates the performance of the present systems and methods against conventional side chain packing methods using the DB379 protein molecule set, in accordance with an embodiment of the present disclosure.

FIG. 7 illustrates the performance of the present systems and methods against conventional side chain packing methods using the CASP-FM 56 template free protein molecule set, in accordance with an embodiment of the present disclosure.

FIG. 8 illustrates the performance of the present systems and methods against conventional side chain packing methods using the CAMEO-Hard protein molecule set, in accordance with an embodiment of the present disclosure.

FIG. 9 illustrates how the present systems and methods exhibits approximately a 5-10° improvement in prediction accuracy for all dihedrals angles across the DB379, CASP-FM 56, and CAMEO-Hard protein molecule sets compared to a prior side packing method termed ZymePack, in accordance with an embodiment of the present disclosure.

FIG. 10 illustrates the side chain dihedral angles of arginine in accordance with the prior art.

Like reference numerals refer to corresponding parts throughout the several views of the drawings.

### DETAILED DESCRIPTION OF THE EMBODIMENTS

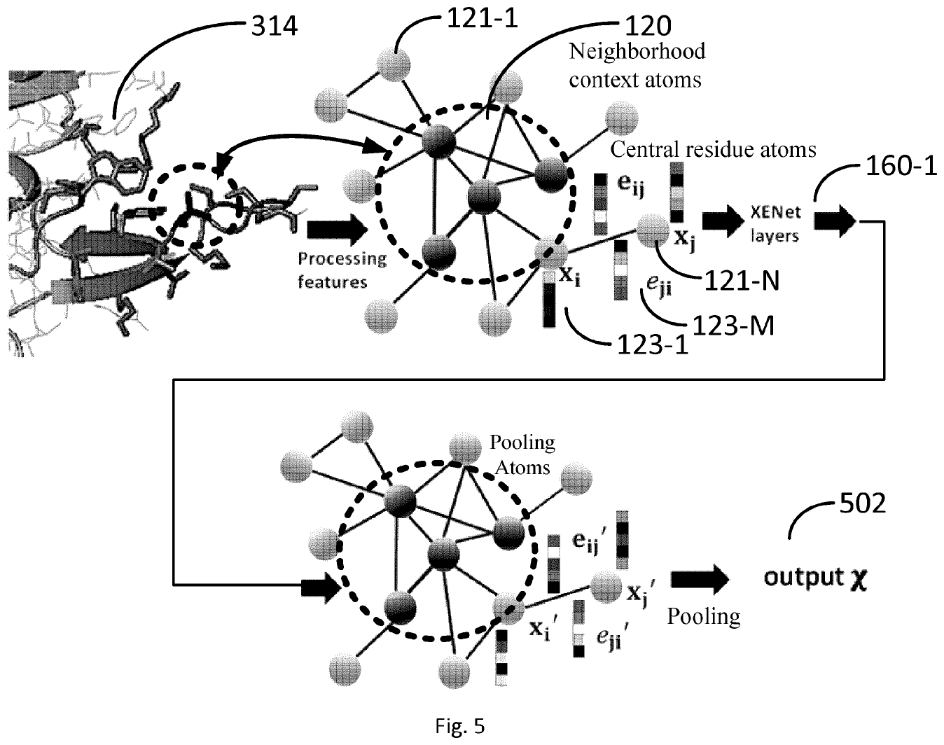

With reference to FIG. 5, the disclosed systems and methods obtain a graph 120 from the atomic coordinates 314 of a target polymer. The graph comprises nodes 121 and edges 123. The nodes represent target polymer atoms. Each respective edge in the plurality of edges encodes information about a corresponding pair of nodes in the plurality of nodes. Such information encoded by the edges includes distances between the corresponding pair of nodes and whether the two atoms collectively represented by the pair of nodes are covalently bound to each other.

In the disclosed systems and methods, the graph is broken up into partial-context subgraphs. Each of these partial-context subgraphs represents a residue in the polymer. Each of the partial-context subgraphs is sequentially inputted into a first model to sequentially calculate, in turn, first side chain dihedral angles 502 for residues of the polymer. The first side chain dihedral angles for residues of the polymer is used to update the graph through the level of the first side chain dihedral angles. In some embodiments the first side chain dihedral angle is the X₁ dihedral angle. Next, each second subgraph in a plurality of second partial-context subgraphs of the updated graph is sequentially inputted into a second model, thereby obtaining calculated second side chain dihedral angles for polymer residues. The second side chain dihedral angles for residues of the polymer is used to once again update the graph, this time through second side chain dihedral angles. In some embodiments, the second side chain dihedral angle is the X₂ dihedral angle.

In embodiments where the polymer is a protein, the first side chain dihedral angle is X₁, and the second side chain dihedral angle is X₂, this partial-context procedure of estimating dihedral angles and updating the graph based on them is repeated for those residues that have a X₃ side chain angle.

In embodiments where the polymer is a protein, the first side chain dihedral angle is X₁, the second side chain dihedral angle is X₂, and the X₃ side chain angles have already been calculated, this partial-context procedure of estimating dihedral angles and updating the graph based on then repeated for those residues that have a X₄ side chain angle.

Once the partial-context procedures have been run to extend the graph all the way through the final side chain dihedral angles using partial-context graphs and models, the extended graph is used to generate a respective full-context subgraph for each residue in all or a substantial portion of the polymer at a target side chain rotamer level. All that is missing in a full-context subgraph at a target side chain rotamer level are those atoms of the target residue that are past a target side chain rotamer level. Thus, for example, if the target side chain rotamer level is X₁ and the polymer is a protein, all that is missing in a respective full-context subgraph are those atoms in the target residue corresponding to the respective full-context subgraph that are beyond the C_{β} carbon. These full-context subgraphs, each such subgraph representing a different residue, are provided to a full-context model. As in the case of the partial-context subgraphs, in the case of proteins, the full-context subgraphs start with a first dihedral angle, such as X₁, and sequentially work out to X₄, in successive iterations of the use of full-context subgraphs and full-context models. In this way, the disclosed systems and methods determine the side-chain rotamers for all or a substantial portion of the residues in a polymer, such as a protein, without reliance on computationally intensive energy functions or extensive side chain rotamer libraries.

As such, the present disclosure provides a computational method/framework for predicting a conformation of a residue side chain using a unique graph representation for polymer structures in which node embeddings comprise of the tuple containing sequence and atom-type descriptions as a single categorical variable, while edge embeddings comprise geometric descriptors that are transformed into standardized, rotational and translational invariant features that are unique to polymer topology and geometry. Transformations employed to the full polymer graph to batch them into local subgraphs allows a neural network to make predictions at the graph level instead of making node level predictions. Moreover, the present disclosure incorporates nodes and edges from atoms that belong to crystal symmetry mates along with the default asymmetric unit to prepare the neighborhood-context subgraph.

The present disclosure makes use of two categories of graph descriptions to at various stages of training and target polymer side-chain prediction. The first description contains partial-context up to the level of the atoms at a hierarchy just below side chain dihedral in question. The second description contain the full-context with all heavy atoms (backbone and sidechain) except for the atoms above the hierarchy for the side chain dihedral angle of the residue in question for the second description.

Moreover, the present disclosure provide a unique training strategy for the models used to predict side chain dihedral angles. In some embodiments, such models are graph based neural networks that each include an embedding layer for the node features, followed by two layers of XENet model (see, for example, the XENet layer disclosed in Maguire et al., 2021, “XENet: Using a new graph convolution to accelerate the timeline for protein design on quantum computers,” PLoS Comput Biol 17(9): e1009037, which is hereby incorporated by reference) with elu activation and augmented by dropout and Batchnorm layers. Following the XENet layers, an average pooling over the nodes of the residue of interest followed by a multilayer perceptron layer with tanh activation to produce the outputs. To train these modes, a novel loss function was developed that is capable of handling ambiguity arising from symmetry in dihedral angle definitions e.g., X₂ of aspartic acid, phenylalanine, and tyrosine, and X₃ of glutamic acid. For unambiguous cases mean squared error (MSE) was used, whereas for the ambiguous cases the error was assigned using the lower of the two losses between the dihedral angle in question, Xᵢ, and Xᵢ−π. A set of partial-context models and a set of full-context models that each have this architecture are trained in accordance with the present disclosure using this loss function. Each partial-context model in the set of partial-context models is for a different side chain dihedral angle. For instance, in the case of proteins, one partial-context model is for X₁ determination, another partial-context model is for X₂ determination, and so forth. Likewise, each full-context model in the set of full-context models is for a different side chain dihedral angle. For instance, in the case of proteins, one full-context model is for X, determination, another full-context model is for X₂ determination, and so forth. Each partial-context model and each full-context model predicts the target side chain dihedral angle of one residue of the polymer at a time.

Moreover, the present disclosure provides a two-step residue acid side chain conformation prediction. The first step entails populating the side chains using the above-described set of partial-context models. This first step starts from the given polymer backbone and works out to the final outermost side chain dihedral angle of each target residue (those residues for which a user has requested side chain angles) in the polymer. Once the first step has elucidated each of the side chain angles of each of the target residues in the polymer, the updated graph from this first step is used as initial input into a second step of iterative refinement using the above described set of full-context models. As in the case of the set of partial-context models, the set of full-context models works iteratively from the backbone to the outermost dihedral angle of each residue in the set of target residues of the polymer.

Advantageously, the local subgraphs that are used by the set of partial-context models and the set of full-context models, when available, incorporate nodes and edges from atoms that belong to crystal symmetry mates along with the default asymmetric unit of the polymers. In such instances, the side chain dihedral angle predictions are made for the asymmetric unit only but at the end of making the prediction during each stage of prediction for the asymmetric unit, the predicted side chain dihedral angles are “mirrored” into the crystal symmetry mates using applicable crystallographic operators.

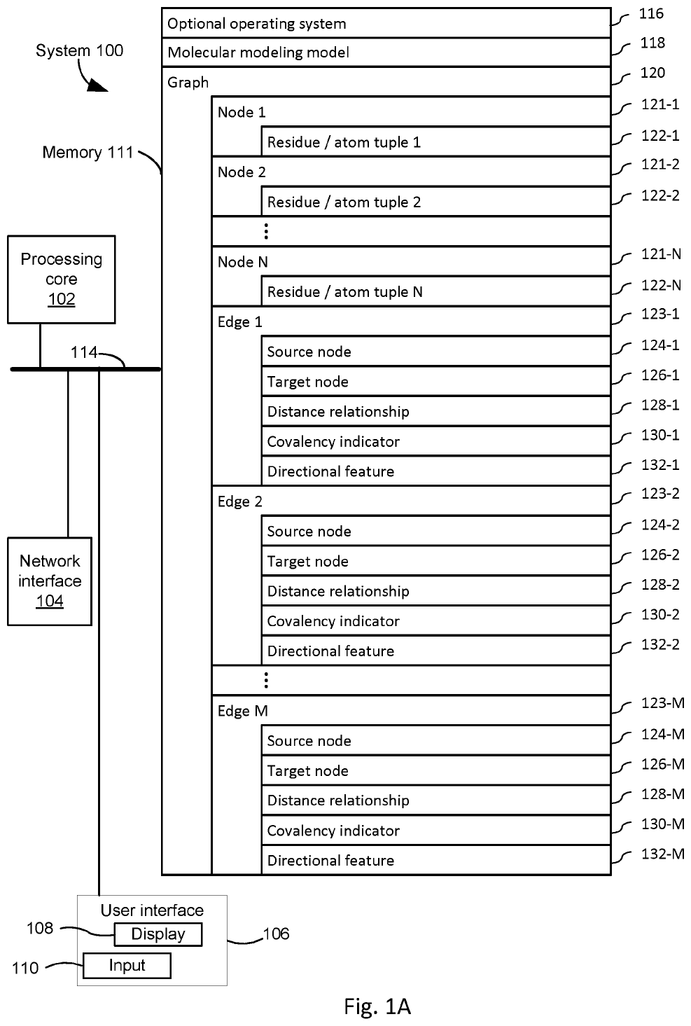

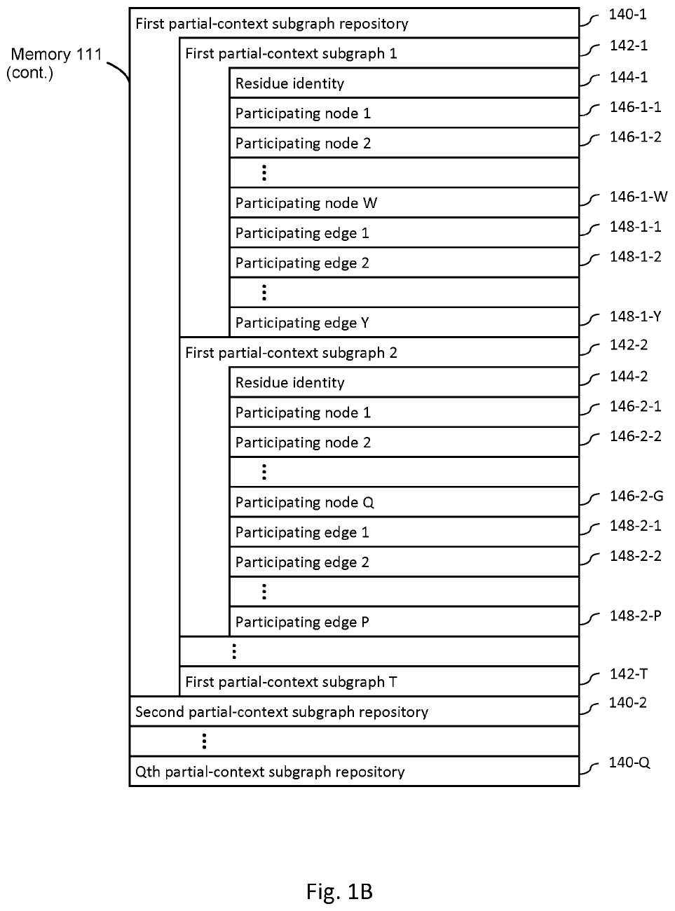

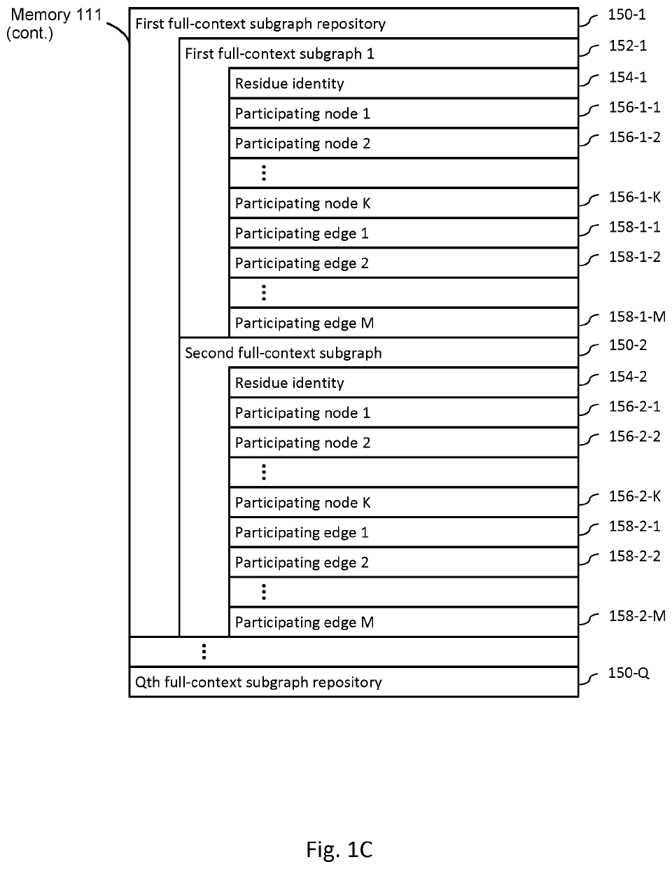

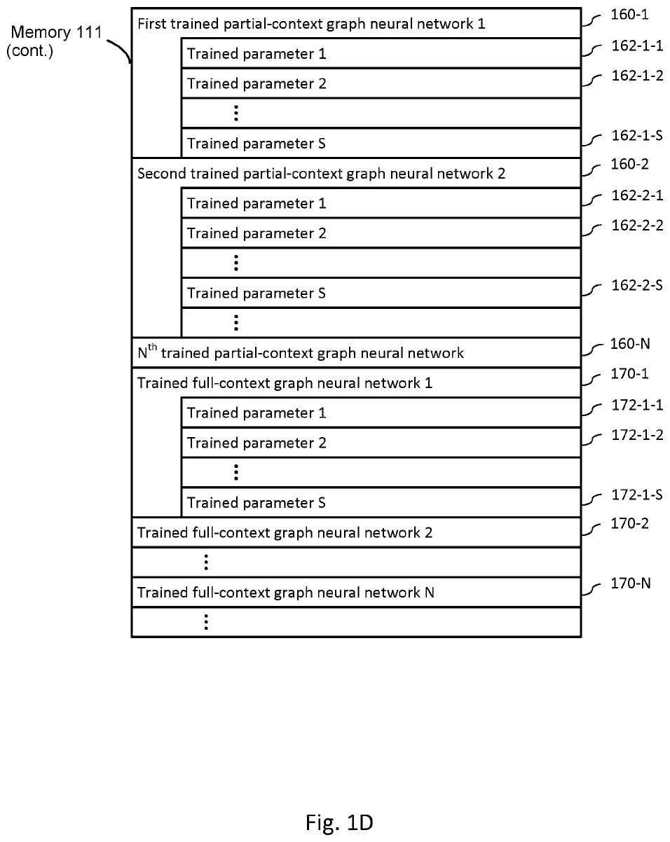

FIG. 1 is a block diagram illustrating a computer system in accordance with the present disclosure. The computer system 100 typically includes one or more processing units (CPUs, sometimes called processors) 102 for executing programs (e.g., programs stored in memory 111), optionally, one or more network or other communications interfaces 104, memory 111, a user interface 106, which includes one or more input devices 110 (such as a keyboard, mouse, keypads, etc.) and one or more output devices such as a display device 108, and one or more communication buses 114 for interconnecting these components. The communication buses 114 may include circuitry (sometimes called a chipset) that interconnects and controls communications between system components.

Memory 111 includes high-speed random access memory, such as DRAM, SRAM, DDR RAM or other random access solid state memory devices; and typically includes non-volatile memory, such as one or more magnetic disk storage devices, optical disk storage devices, flash memory devices, or other non-volatile solid state storage devices. Memory 111 optionally includes one or more storage devices remotely located from the CPU(s) 102. Memory 111, or alternately the non-volatile memory device(s) within memory, comprises a non-transitory computer readable storage medium. In some embodiments, memory 111 or the computer readable storage medium of memory stores the following programs, modules and data structures, or a subset thereof:
an optional operating system 116 that includes procedures for handling various basic system services and for performing hardware dependent tasks;
an optional communication module 741 that is used for connecting the computer 710 to other computers via the one or more communication interfaces 720 (wired or wireless) and one or more communication networks 734, such as the Internet, other wide area networks, local area networks, metropolitan area networks, and so on;
a molecular modeling module 118 that includes instructions for determining the rotamer angles of sidechains of a polymer;
a graph 120 of at least a portion of a polymer, where the graph comprises a plurality of nodes 121 and a plurality of edges 123, each node in the plurality of nodes representing an atom of the polymer (e.g., as a residue/atom tuple 122), and each respective edge 123 in the plurality of edges corresponding to a source node 124 and target node 126 in the plurality of nodes, a distance relationship 128 between the source and target node, a covalency indicator 130 specifying whether the atom associated with the corresponding source node is covalently bound to the atom associated with the corresponding destination note, and a directional feature 132 associated with the source and target node;
a first partial-subgraph repository 140-1, first partial-subgraph repository 140-1, through Q^{th} partial-subgraph repository 140-Q, each partial-subgraph repository comprising a corresponding plurality of partial-context subgraphs 142 of the graph, each respective partial-context subgraph corresponding to a respective residue of the polymer and drawn from the nodes in the graph that represent atoms of the respective residue before a designated side chain dihedral angle, or atoms of the polymer proximate to the respective residue and therefore including the respective residue identity 144, each participating node 146, and each participating edge 148;
a first full-context subgraph repository 150-1 through a Q^{th} (150-Q) full-context subgraph repository, where Q again is a positive integer of 2 or greater, each respective full-context subgraph repository comprising a plurality of full-context subgraphs 152 of the graph, each respective full-context subgraph corresponding to a respective residue of the protein and drawn from the nodes in the graph that represent atoms of the respective residue before a target side chain dihedral angle or all atoms of the polymer other than the respective residue and therefore including the respective residue identity 154, each participating node 156, and each participating edge 158;
a first (160-1) through N^{th} (160-N) trained partial-context graph neural network, where N is a positive integer of 2 or greater, each respective partial-context graph neural network 160 comprising a plurality of parameters 162; and
a first (170-1) through N^{th} (170-N) trained full-context graph neural network, where N is again a positive integer of 2 or greater, each respective full-context graph neural network 170 comprising a plurality of parameters 172.

In some implementations, one or more of the above identified data elements or modules of the computer system 100 are stored in one or more of the previously mentioned memory devices, and correspond to a set of instructions for performing a function described above. The above identified data, modules or programs (e.g., sets of instructions) need not be implemented as separate software programs, procedures or modules, and thus various subsets of these modules may be combined or otherwise re-arranged in various implementations. In some implementations, the memory 111 optionally stores a subset of the modules and data structures identified above. Furthermore, in some embodiments, the memory 111 stores additional modules and data structures not described above.

As illustrated in FIG. 1, the disclosed systems and method make use of two categories of models, partial-context (PC) models 160-1, . . . , 160-N, and full-context (FC) models 170-1, . . . , 170-N, respectively, where N is a positive integer of 2 or greater. In some embodiments, the polymer is a protein, N is four, and each category of models includes a separate model for each of the possible side chain dihedral angles, X₁, X₂, X₃, and X₄, found in naturally occurring amino acids. While the general approach and network architecture for models of both categories, PC and FC, are the same, the amount of information used to build the protein graphs for these models is characteristically different.

The graphs used for training PC models for a given dihedral angle are constructed using all the heavy (non-hydrogen) backbone atoms and side chain atoms up to the given side chain dihedral angle. Thus, in the case where the polymer is a protein, a first PC model 160-1 is trained up to X₁ for every standard residue in the protein, or at least those residues that have been targeted for side chain optimization, that has a X₁ side chain dihedral angle. In the case of this first PC model, a first graph adjacency matrix is created for each protein by limiting to a cut off number (e.g., 40) of nearest neighbors to each residue, e.g., the backbone atoms and side chain atoms, in the case of residues other than glycine, up to X₁. A second PC model 160-2 is trained up to X₂ for every standard residue in the protein, or at least those residues that have been targeted for side chain optimization, that has a X₂ side chain dihedral angle. In the case of this second PC model, a second graph adjacency matrix is created for each protein by limiting to a cut off number (e.g., 40) of nearest neighbors to each residue, e.g., the backbone atoms and side chain atoms up to X₂. A third PC model 160-3 is trained up to X₃ for every standard residue in the protein, or at least those residues that have been targeted for side chain optimization, that has a X₃ side chain dihedral angle. In the case of this third PC model, a third graph adjacency matrix is created for each protein by limiting to a cut off number (e.g., 40) of nearest neighbors to each residue, e.g., the backbone atoms and side chain atoms up to X₃. Finally, a fourth PC model 160-4 is trained up to X₄ side chain dihedral angle for every standard residue in the protein, or at least those residues that have been targeted for side chain optimization, that has a X₄ side chain dihedral angle. In the case of this fourth PC model, a graph adjacency matrix is created for each protein by limiting to a cut off number (e.g., 40) of nearest neighbors to each residue, e.g., the backbone atoms and side chain atoms. As such, for the PC models, instead of using the full graph for the entire protein, the graph data is transformed and batched into “local” subgraphs for each residue within the protein such that the nodes within each subgraph contain the union of all atoms within the residue and their cut off number (e.g., 40) of nearest neighbors.

For FC models, on the other hand, the graphs are constructed using the backbone atoms and side chain atoms up to the subject side chain dihedral angle for the residue in question and all backbone and side chain atoms for all other residues in that protein. Thus, in the case where the polymer is a protein, a first FC model 170-1 is trained up to X₁ for every standard residue in the protein, or at least those residues that have been targeted for side chain optimization, that has a X₁ side chain dihedral angle. In the case of this first FC model, a first graph adjacency matrix is created for each protein using all atoms of the protein other than the atoms up to the X₁ side chain dihedral angle of the target residue. Like the PC models, a different FC model is created for each level of side chain dihedral angle which, in the case of proteins is, X₁, X₂, X₃, and X₄.

In some embodiments, the node 120 attributes of the graphs 120 are categorical variables created using the (residue name, atom name) tuple 124.

In some embodiments, the edge 122 attributes are bidirectional and comprise three types of standardized features, a) a pairwise distance 128, b) a direction vector between two nodes in the graph 132, and c) a covalency indicator 130 (e.g., a binary input) to distinguish between covalently bonded atoms and otherwise.

In some embodiments, the pairwise distance 128 rᵢⱼ between the source node 124 and target node 126 of an edge 122 is embedded as e^{-r}^{ᵢⱼ}^{²}^{/κ}, where x is the square of 10 Å (the standard cutoff distance for nonbonded interactions), i is the identity of the source node 124, j is the identity of the target node 126, and rᵢⱼ is the distance between the atom represented by the source node (i) and the atom represented by the target node 126.

The direction vector between two nodes in the graph 132 serves to account for apparent anisotropy in relative placement inside the protein and is, in some embodiments, computed using local coordinate frame constructed using coordinates of atoms covalently bonded to the atom in question.

Accurate prediction of side-chain conformation is an important component of protein structure and function optimization. In single-site mutants and in homologous proteins, the backbone conformation change is minimal and accurate side chain conformation prediction is sufficient for structure refinement. For in-silico structure refinement that includes backbone conformation change, one stage in the refinement process is prediction, e.g. side chain repacking. Accurate knowledge of side chain geometry is important for protein binding site recognition, in-silico binding affinity assessment, and for interface engineering between cognate binding proteins and protein/ligand complexes. For the protein design problem, one needs to co-optimize changes in the sequence along with backbone and side chain conformations.

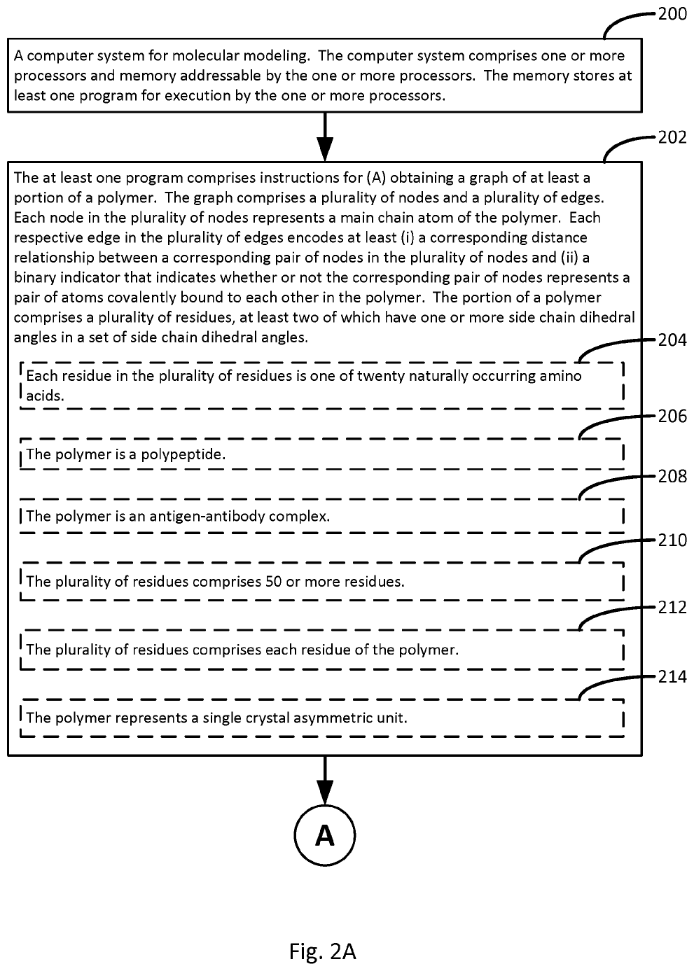

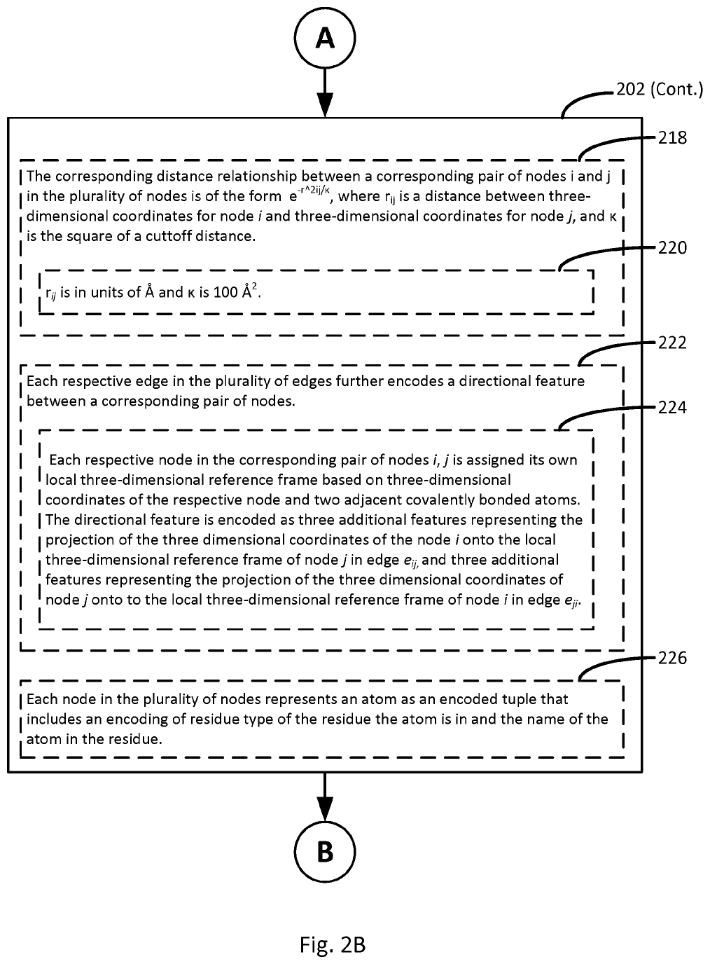

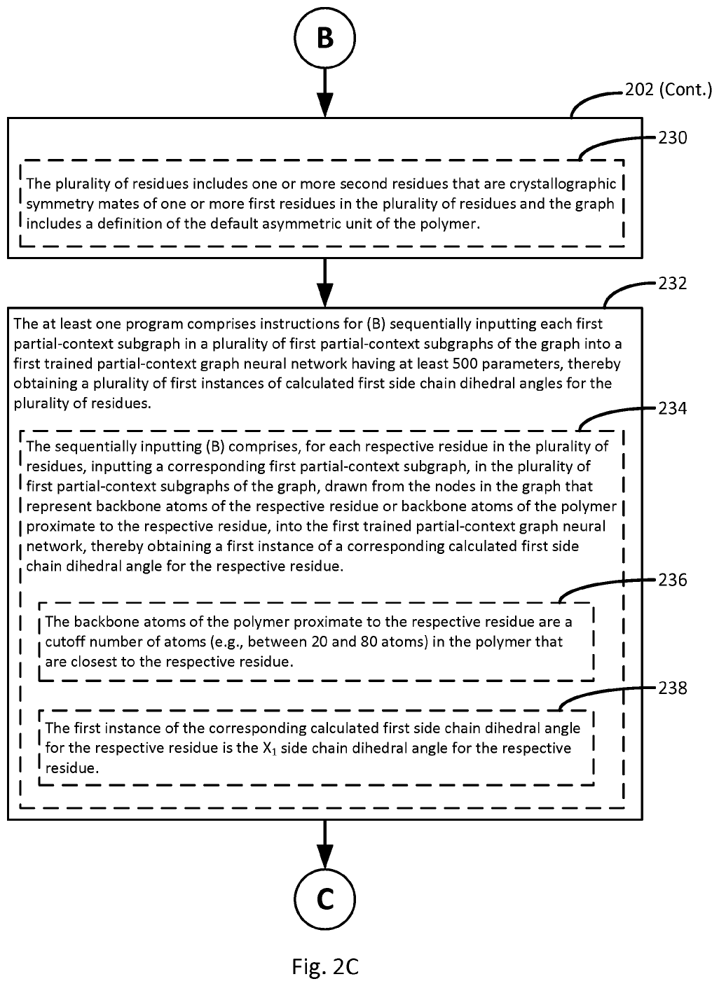

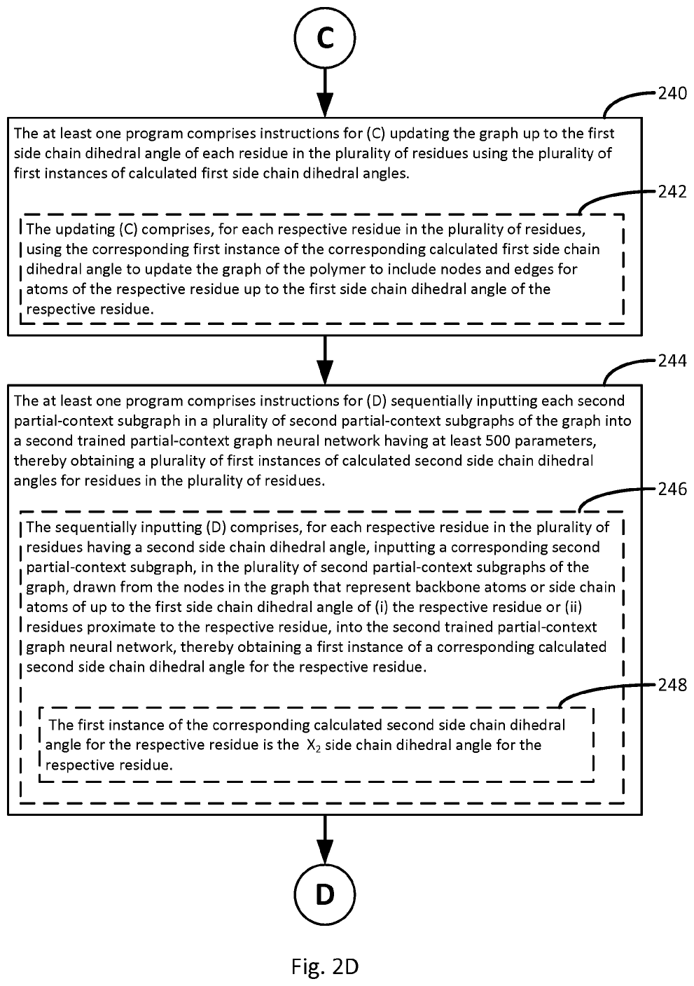

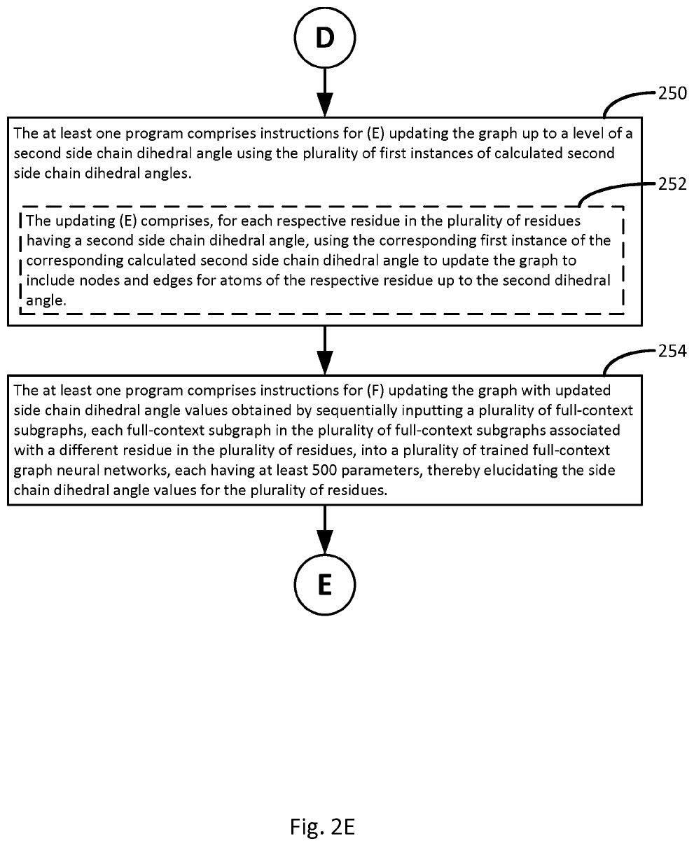

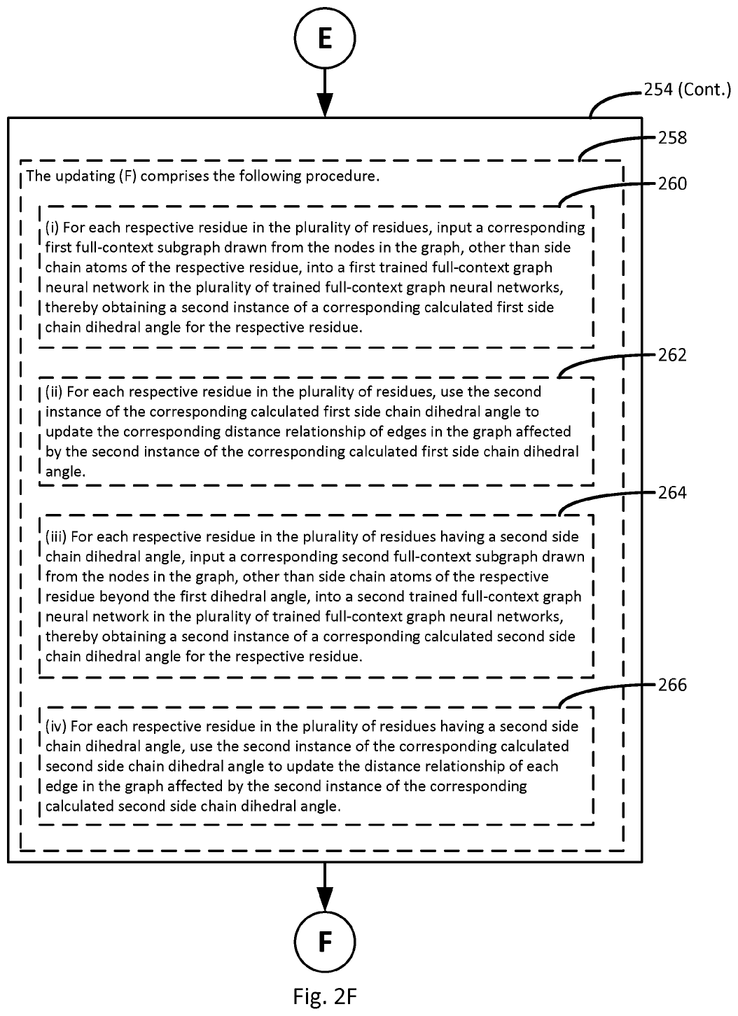

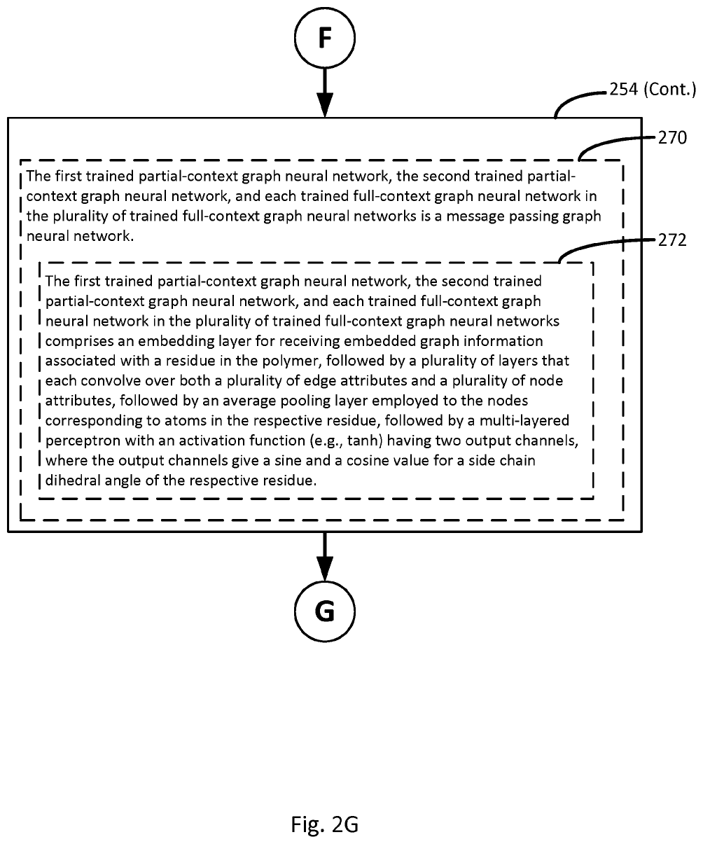

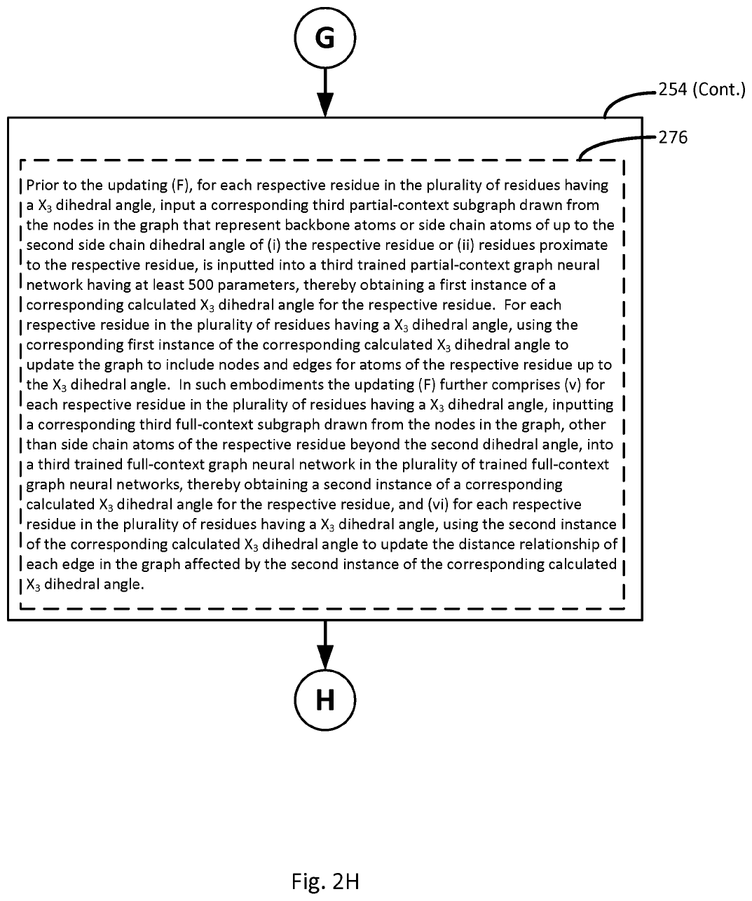

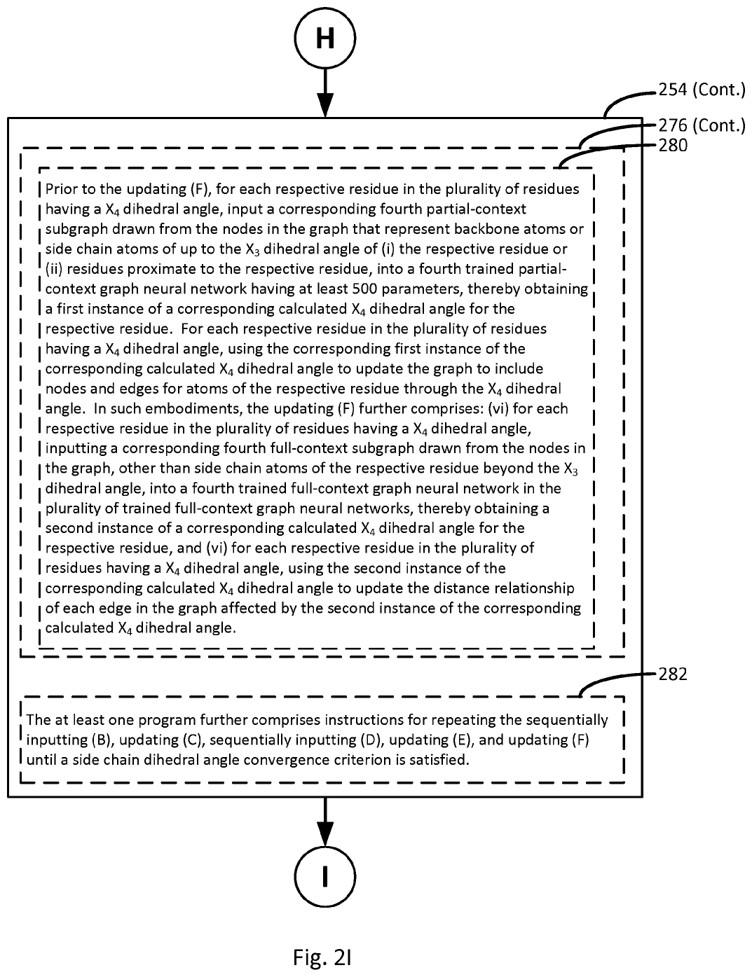

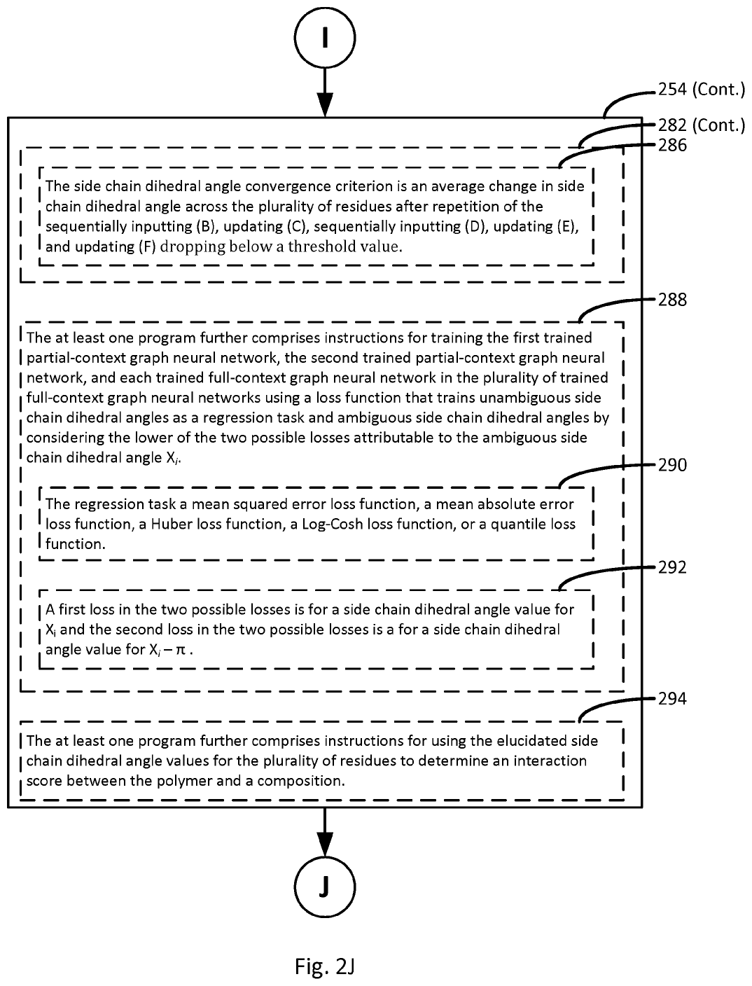

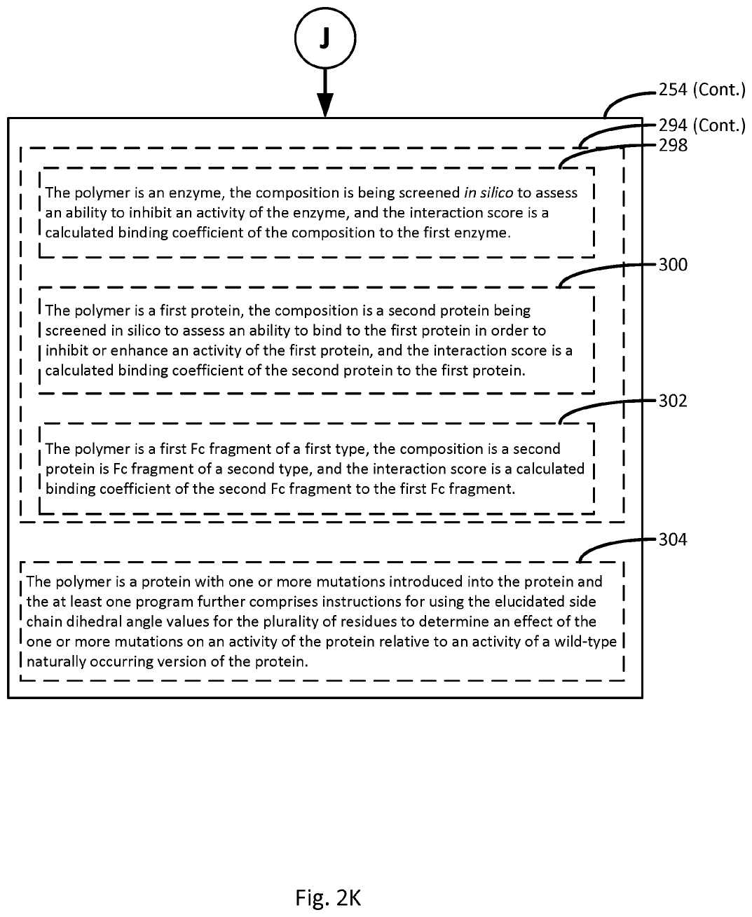

Now that a system for molecular modeling (e.g., through side chain packing) has been generally disclosed, methods for performing such characterization is detailed with reference to FIG. 2 and discussed below.

Block 200. Referring to block 200 of FIG. 2A, a computer system 100 for molecular modeling is provided. The computer system comprises one or more processors and memory addressable by the one or more processors. The memory stores at least one program for execution by the one or more processors. For instance in some embodiments the at least one program is molecular modeling model 188 of FIG. 1A.

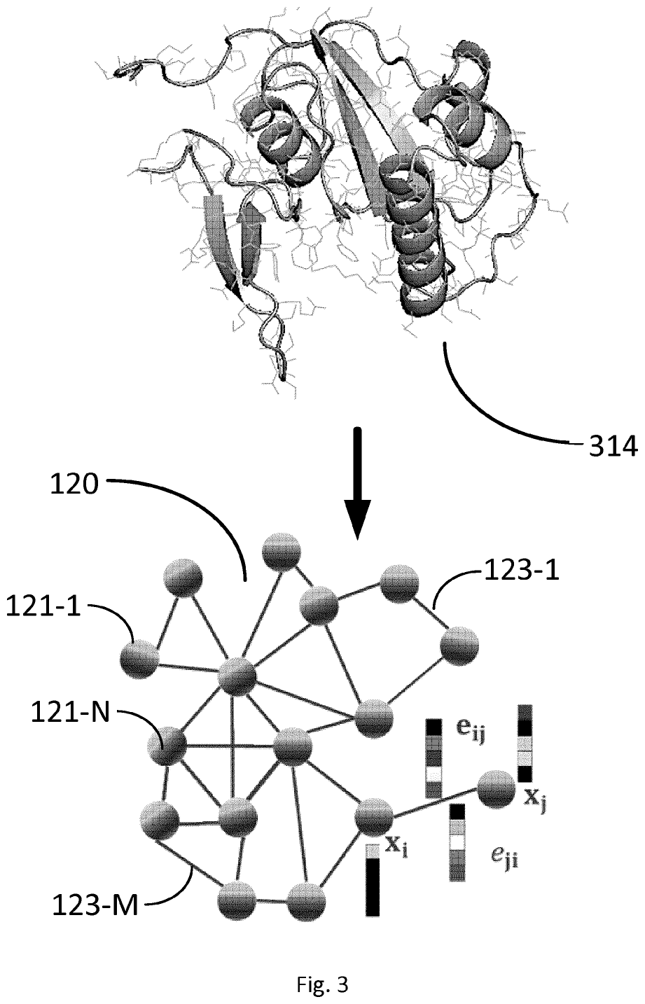

Blocks 202-214. Referring to block 202 of FIG. 2A, the at least one program comprises instructions for (A) obtaining a graph 120 of at least a portion of a polymer. This portion of the polymer comprises a plurality of residues. The graph 120 comprises a plurality of nodes 121 and a plurality of edges 123. Initially in the graph, each node 121 in the plurality of nodes represents a main chain atom of the polymer. FIG. 3 illustrates. In FIG. 3, the target polymer, for example in the form of an atomic structure 314 of the polymer, is converted into a graph representation 120 with nodes 121 representing atoms and geometric relations with neighboring atoms represented by edges 123. Advantageously, the graph representation is rotationally/translationally invariant by construction.

In some embodiments, the atomic model of the polymer that is used to construct the graph 120 is the set of M three-dimensional coordinates {x₁, . . . , x_{M}}, where the term M here is a positive integer that is indexed across either all atoms, or all heavy (non-hydrogen) atoms of the polymer. Thus, in some embodiments, the set of M three-dimensional coordinates {x₁, . . . , x_{M}} for the polymer include coordinates of all backbone atoms in the polymer other than hydrogen atoms. In some embodiments, the set of M three-dimensional coordinates {x₁, . . . , x_{M}} for the polymer includes coordinates of all backbone atoms in the polymer including hydrogen atoms. In some embodiments, these coordinates are obtained by x-ray crystallography, nuclear magnetic resonance spectroscopic techniques, or electron microscopy. In some embodiments, the set of M three-dimensional coordinates {x₁, . . . , x_{M}} is obtained by modeling (e.g., molecular dynamics simulations, homology modeling, etc.). In typical embodiments, each coordinate in {x₁, . . . , x_{N}} is a relative Cartesian coordinate in three dimensional space (e.g., x, y z). In some embodiments, there are ten or more, twenty or more, thirty or more, fifty or more, one hundred or more, between one hundred and five thousand, or less than 500 residues in the polymer 44. In some embodiments, the set of M three-dimensional coordinates {x₁, . . . , x_{M}} for the polymer also includes coordinates of side chains that are not in the plurality of residues that are being optimized by the systems and methods of the present disclosure. In embodiments in which the first trained-partial context neural network 160-1 optimizes X₁ side chain dihedral angles and the polymer is a protein, application of the first trained-partial-context graph neural network 160-1, as discussed below in conjunction with block 230, requires that the set of M three-dimensional coordinates include the coordinates of the C_{β} atom of each residue in the plurality of residues of the polymer that are to be optimized. In some embodiments, given a protein backbone, the side chain C_{β} atom in each target residue side chain is first deterministically predicted using a conventional C_{β} residue builder tool based on the coordinates of the backbone atoms of the polymer in the set of M three-dimensional coordinates {x₁, . . . , x_{M}}. Once the coordinates of the C_{β} atom for the target residue are populated, they included in the graph 102 and respective subgraphs 142/152 derived from the graph.

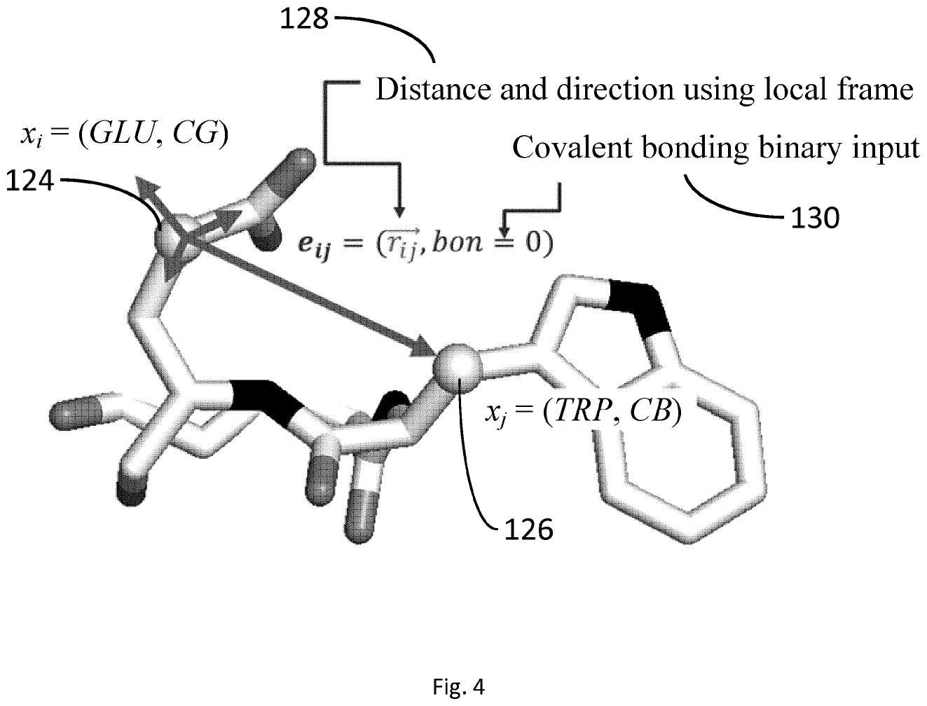

As discussed below, in later stages, the graph 120 is expanded to include nodes 121 for side chain atoms. With the exception of the C_{β} atom as discussed, above, this involves elucidating the atomic coordinates of the side chain atoms using the disclosed models, rather than obtaining such coordinates from the starting set of M three-dimensional coordinates {x₁, . . . , x_{M}}. As illustrated in FIG. 4, each respective edge in the plurality of edges encodes at least (i) a corresponding distance relationship 128 between a corresponding pair of nodes (e.g., corresponding source node 124 and corresponding target node 126) in the plurality of nodes and (ii) a binary indicator 130 that indicates whether or not the corresponding pair of nodes represents a pair of atoms covalently bound to each other in the polymer. For example, consider the case where an edge represents the C_{α} and C_{β} atoms within a single residue. In this instance, the covalency indicator 130 will have a first value (e.g., “1”) to indicate that the two atoms are covalently bound to each other. As another example, consider the case where an edge represents the C_{α} atom of a first residue and the C_{β} atom of a neighboring residue within the polymer. In this instance, the covalency indicator 130 will have a second value, different from the first value, (e.g., “0”) to indicate that the two atoms are not covalently bound to each other.

The portion of the polymer referenced in block 202 comprises a plurality of residues, at least two of which have one or more side chain dihedral angles in a set of side chain dihedral angles. More typically, in some embodiments, the plurality of residues of the polymer (those for which side chain conformations are elucidated in accordance with the present disclosure) comprises at least 10, 20, 30, 40, 50, 60, 70, 80, 90, or 95 percent of the residues of the polymer, each of which has one or more side chain dihedral angles. In some embodiments, the plurality of residues comprises at least 10, 15, 20, 25, 30, 35, 40, 45, 50, 55, 75, 100, 200, 300, 400 or more residues each of which has one or more side chain dihedral angles.

Referring to block 204, in some embodiments, the polymer is a protein and each residue in the plurality of residues is one of twenty naturally occurring amino acids: alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine. In some embodiments, the polymer is a protein and each residue in the plurality of residues is one of the twenty naturally occurring amino acids that has at least one side chain dihedral angle. Thus, in such embodiments, the plurality of residues does not include glycine or alanine. However, the protein can include glycine and alanine in such embodiments. In some embodiments, the disclosed systems and methods are extended to predict the side chain dihedral angles of modifications of the twenty naturally occurring amino acids, such as 2-aminoadipic acid, 3-aminoadipic acid, 2-aminobutyric acid, 4-aminobutyric acid, 6-aminocaproic acid, 2-aminoheptanoic acid, 2-aminoisobutyric acid, 3-aminoisobutyric acid, 2-aminopimelic acid, 2,4 diaminobutyric acid, desmosine, 2,2′-diaminopimelic acid, 2,3-diaminopropionic acid, N-ethylglycine, N-ethylasparagine, hydroxylysine, allo-hydroxylysine, 3-hydroxyproline, 4-hydroxyproline, isodesmosine, allo-isoleucine, N-methylglycine, N-methylisoleucine, 6-N-methyllysine, N-methylvaline, norvaline, norleucine, and/or ornithine, to name some non-limiting examples. In some embodiments, these non-standard amino acids are evaluated as their closest normally occurring amino acid during calculation of the model and are then converted back to their non-standard amino acid once the model has been completed.

Referring to block 206, in some embodiments, the polymer is a polypeptide. Referring to block 208, in some embodiments, the polymer is an antigen-antibody complex.

Referring to block 210, in some embodiments the plurality of residues, that is, the number of residues in the polymer that the discloses systems and methods will concurrently determine the side chain torsion angles for, comprises 50 or more residues. More generally, in some embodiments the plurality of residues of the polymer comprises at least 10, 20, 30, 40, 50, 60, 70, 80, 90, or 95 percent of the residues of the polymer, each of which has side chain dihedral angles. In some embodiments, the plurality of residues comprises at least 10, 15, 20, 25, 30, 35, 40, 45, 50, 55, 75, 100, 200, 300, 400 or more residues. Elucidation of the side chain torsion angles for these residues, together with the obtained M three-dimensional coordinates {x₁, . . . , x_{M}} for the backbone atoms of the polymer results in the elucidation of the atomic coordinates of each side chain in the plurality of side chains. In some embodiments, the plurality of residues represent more than one contiguous region of the polymer, such as exposed loops of the polymer. In some embodiments, only solvent exposed residues of the polymer are selected for side chain conformational refinement. There is no requirement that the plurality of residues be contiguous in the sequence of polymer.

Referring to block 212 of FIG. 2A, in some embodiments, the plurality of residues comprises each residue of the polymer. In some embodiments, the plurality of residues of the polymer comprises at least 10, 20, 30, 40, 50, 60, 70, 80, 90, or 95 percent of the residues of the polymer.

Referring to block 214, in some embodiments, the polymer represents a single crystal asymmetric unit. In such embodiments, the set of M three-dimensional coordinates {x₁, . . . , x_{M}} includes only those coordinates of the polymer that are in a single crystallographic asymmetric unit. In other embodiments, local subgraphs used in the present disclosure incorporate, when available, nodes and edges from atoms of the target polymer to a plurality of atoms that belong to crystal symmetry mates outside the asymmetric unit along with the atoms of the polymer within the default asymmetric unit. In such instances, the side chain dihedral angle predictions are made for the asymmetric unit only but at the end of making the prediction during each stage of prediction for the asymmetric unit, the predicted side chain dihedral angles are “mirrored” into the crystal symmetry mates using applicable crystallographic operators.

In some embodiments, the polymer comprises between 2 and 5,000 residues, between 20 and 50,000 residues, more than 30 residues, more than 50 residues, or more than 100 residues. In some embodiments, a residue in the polymer comprises two or more atoms, three or more atoms, four or more atoms, five or more atoms, six or more atoms, seven or more atoms, eight or more atoms, nine or more atoms or ten or more atoms. In some embodiments the polymer has a molecular weight of 100 Daltons or more, 200 Daltons or more, 300 Daltons or more, 500 Daltons or more, 1000 Daltons or more, 5000 Daltons or more, 10,000 Daltons or more, 50,000 Daltons or more or 100,000 Daltons or more.

A polymer, such as those that can be studied using the disclosed systems and methods, is a large molecular system composed of repeating structural units. These repeating structural units are termed particles or residues interchangeably herein. In some embodiments, each particle pᵢ in the set of {p₁, . . . , p_{K}} particles represents a single different residue in the native polymer. To illustrate, consider the case where the native comprises 100 residues. In this instance, the set of {p₁, . . . , p_{K}} comprises 100 particles, with each particle in {p₁, . . . , p_{K}} representing a different one of the 100 particles, and k is a positive integer of 2 or greater, 3 or greater, 10 or greater, 20 or greater, or between 30 and 10,000.

In some embodiments, the polymer that is evaluated using the disclosed systems and methods is a natural material in which at least some of the residues of the natural material have one or more dihedral angles. In some embodiments, the polymer is any synthetic material in which at least some of the residues of the synthetic material have one or more dihedral angles.

In some embodiments, the polymer is a polypeptide. As used herein, the term “polypeptide” means two or more amino acids or residues linked by a peptide bond. The terms “polypeptide” and “protein” are used interchangeably herein and include oligopeptides and peptides. An “amino acid,” “residue” or “peptide” refers to any of the twenty standard structural units of proteins as known in the art. The designation of an amino acid isomer may include D, L, R and S. The definition of amino acid includes nonnatural amino acids. Thus, selenocysteine, pyrrolysine, lanthionine, 2-aminoisobutyric acid, gamma-aminobutyric acid, dehydroalanine, ornithine, citrulline and homocysteine are all considered amino acids. Other variants or analogs of the amino acids are known in the art. Thus, a polypeptide may include synthetic peptidomimetic structures such as peptoids. See Simon et al., 1992, Proceedings of the National Academy of Sciences USA, 89, 9367, which is hereby incorporated by reference herein in its entirety. See also Chin et al., 2003, Science 301, 964; and Chin et al., 2003, Chemistry & Biology 10, 511, each of which is incorporated by reference herein in its entirety.

In some embodiments, the polypeptides evaluated in accordance with some embodiments of the disclosed systems and methods may also have any number of posttranslational modifications. Thus, a polypeptide includes those that are modified by acylation, alkylation, amidation, biotinylation, formylation, 7-carboxylation, glutamylation, glycosylation, glycylation, hydroxylation, iodination, isoprenylation, lipoylation, cofactor addition (for example, of a heme, flavin, metal, etc.), addition of nucleosides and their derivatives, oxidation, reduction, pegylation, phosphatidylinositol addition, phosphopantetheinylation, phosphorylation, pyroglutamate formation, racemization, addition of amino acids by tRNA (for example, arginylation), sulfation, selenoylation, ISGylation, SUMOylation, ubiquitination, chemical modifications (for example, citrullination and deamidation), and treatment with other enzymes (for example, proteases, phosphotases and kinases). Other types of posttranslational modifications are known in the art and are also included.

Blocks 218-220. Referring to block 218 of FIG. 2B, in some embodiments the corresponding distance relationship between a corresponding pair of nodes i and j in the plurality of nodes is of the form e^{-r}^{ᵢⱼ}^{²}^{/κ}, where rᵢⱼ is a distance between three-dimensional coordinates for node i and three-dimensional coordinates for nodeji and κ is the square of a nonbonded cutoff distance. Referring to block 220, in some embodiments the value rᵢⱼ is in units of Å and κ is 100 Å². In some embodiments, K is between 50 Å² and 200 Å², such as 50 Å², 60 Å², 70 Å², 80 Å², 90 Å², 100 Å², 110 Å², 120 Å², 130 Å², 140 Å², 150 Å², 160 Å², 170 Å², 180 Å², 190 Å², or 200 Å².

Blocks 222-224. Referring to block 222 of FIG. 2B, each respective edge in the plurality of edges further encodes a directional feature 132 between a corresponding pair of nodes. Referring to block 224, each respective node in the corresponding pair of nodes is assigned its own local three-dimensional reference frame based on three-dimensional coordinates of the respective node and two adjacent covalently bonded atoms. The directional feature is encoded as three additional features representing the projection of the three dimensional coordinates of the first node in the corresponding pair of nodes onto to the local three-dimensional reference frame of the second node in the corresponding pair of nodes. In some embodiments, each respective edge in the plurality of edges encodes the directional feature between a corresponding pair of nodes. For example, in some such embodiments, for each of pair of atoms (e.g., or equivalently each pair of nodes) i and j in the graph, there are two different edges, eᵢⱼ which is from i to j, and eⱼᵢ, which is from j to i. In such embodiments, both eᵢⱼ and eⱼᵢ include five features, two of which are the same. The first feature is distance (a corresponding distance relationship between i and j), as discussed above, which is the same for eᵢⱼ and eⱼᵢ. The second feature is a binary indicator that indicates whether or not i and j are covalently bound to each other in the polymer, as discussed above, which is the same for eᵢⱼ and eⱼᵢ. The remaining three features eᵢⱼ and eⱼᵢ encode the directional vector from atom i to atom j, in the case of eⱼᵢ and the directional vector from atom j to atom i, in the case of eⱼᵢ. Thus, there is a representation of 1×5 for each direction in the bidirectional graph. The directional vector (encoded as the final three features) for eᵢⱼ and eⱼᵢ is specific to the direction based upon the local coordinate system placed on i and similarly on]. In some embodiments, to do this, a local reference frame using an orthonormal basis, \vec{eₓ}, \vec{e_{y}}, \vec{e_{Z}} is placed at each atom in question, B, which is calculated from the three-dimensional coordinates of the two adjacent bonded atoms A, C and the atom in question B. There are multiple ways to use A, B and C to define a coordinate system. For example, in some embodiments, A, B, and C define a coordinate system in the manner described in Sverrisson et al., “Fast end-to-end learning on protein surfaces,” https://www.biorxiv.org/content/10.1101/2020.12.28.424589v1.full.pdf, which is hereby incorporated by reference.

As another example, in some embodiments

$$\vec{{e}_{Z}} = \frac{\vec{( AB )} - \vec{( BC )}}{\vert \vec{( AB )} - \vec{( BC )} \vert}$$

$$\vec{{e}_{x}} = \frac{\vec{( BC )} - \vec{( AB )}}{\vert \vec{( BC )} - \vec{( AB )} \vert}$$

$$and \vec{{e}_{y}} = \vec{{e}_{z}} - \vec{{e}_{x}} .$$

In such embodiments, the identity of atoms A and C for each atom in the twenty naturally occurring amino acids is set forth in Table 1 below.

TABLE 1

| Residue Name | B | A | C | Stage |
|---|---|---|---|---|
| all | N | C*¹ | CA*¹ | Initial |
| | CA | N | C*¹ | Initial |
| | C | CA | N | Initial |
| | O | C | CA | initial |
| all and not GLY | CB | CA | N | chi_1² |
| ARG | CG | CB | CA | chi_2² |
| | CD | CG | CB | chi_3² |
| | NE | CD | CG | chi_4² |
| | CZ | NE | CD | final³ |
| | NH1 | CZ | NE | final³ |
| | NH2 | CZ | NE | final³ |
| ASN | CG | CB | CA | chi_2 |
| | ND2 | CG | CB | chi_3 |
| | OD1 | CG | CB | chi_4 |
| ASP | CG | CB | CA | chi_2 |
| | OD2 | CG | CB | chi_3 |
| | OD1 | CG | CB | chi_3 |
| CYS | SG | CB | CA | chi_2 |
| GLN | CG | CB | CA | chi_2 |
| | CD | CG | CB | chi_3 |
| | OE1 | CD | CG | chi_4 |
| | NE2 | CD | CG | chi_4 |
| GLU | CG | CB | CA | chi_2 |
| | CD | CG | CB | chi_3 |
| | OE1 | CD | CG | chi_4 |
| | OE2 | CD | CG | chi_4 |
| HIS | CG | CB | CA | chi_2 |
| | ND1 | CG | CB | chi_3 |
| | CD2 | CG | CB | chi_3 |
| | CE1 | ND1 | CG | chi_4 |
| | NE2 | CD2 | CG | chi_4 |
| ILE | CG1 | CB | CA | chi_2 |
| | CG2 | CB | CA | chi_2 |
| | CD1 | CG1 | CB | chi_3 |
| LEU | CG | CB | CA | chi_2 |
| | CD1 | CG | CB | chi_3 |
| | CD2 | CG | CB | chi_3 |
| LYS | CG | CB | CA | chi_2 |
| | CD | CG | CB | chi_3 |
| | CE | CD | CG | chi_4 |
| | NZ | CE | CD | final |
| MET | CG | CB | CA | chi_2 |
| | SD | CG | CB | chi_3 |
| | CE | SD | CG | chi_4 |
| PHE | CG | CB | CA | chi_2 |
| | CD1 | CG | CB | chi_3 |
| | CD2 | CG | CB | chi_3 |
| | CE1 | CD1 | CG | chi_4 |
| | CE2 | CD1 | CG | chi_4 |
| | CZ | CE1 | CD1 | chi_4 |
| PRO | CG | CB | CA | chi_2 |
| | CD | CG | CB | chi_3 |
| SER | OG | CB | CA | chi_2 |
| TRP | CG | CB | CA | chi_2 |
| | CD1 | CG | CB | chi_3 |
| | CD2 | CG | CB | chi_3 |
| | NE1 | CD1 | CG | chi_4 |
| | CE2 | CD2 | CG | chi_4 |
| | CE3 | CD2 | CG | chi_4 |
| | CZ2 | CE2 | NE1 | chi_4 |
| | CZ3 | CE3 | CD2 | chi_4 |
| | CH2 | CZ2 | CE2 | chi_4 |
| THR | OG1 | CB | CA | chi_2 |
| | CG2 | CB | CA | chi_2 |
| TYR | CG | CB | CA | chi_2 |
| | CD1 | CG | CB | chi_3 |
| | CD2 | CG | CB | chi_3 |
| | CE1 | CD1 | CG | chi_4 |
| | CE2 | CD2 | CG | chi_4 |
| | CZ | CE1 | CD1 | chi_4 |
| | OH | CZ | CE1 | chi_4 |
| VAL | CG1 | CB | CA | chi_2 |
| | CG2 | CB | CA | chi_2 |

¹C* and CA* refer to the carbon and the Cₐ carbon of the previous residue, i.e. the residue preceding on the N terminus of the residue of interest
²Needed before predicting
³After chi_4 is predicted.

Block 226. Referring to block 226, in some embodiments each node 121 in the plurality of nodes represents an atom as an encoded tuple that represents both the residue type of the residue the atom is in and the name of the atom. For instance, in some embodiments, in the case where the polymer is a protein and just the twenty naturally occurring amino acids are considered, each heavy atom in the 20 standard residue types is treated separately, resulting in 167 atom types.

Block 230. Referring to block 230 of FIG. 2C, in some embodiments, the plurality of residues includes one or more second residues that are crystallographic symmetry mates of one or more first residues in the plurality of residues and the graph includes a definition of the default asymmetric unit of the polymer. As detailed in Example 2 below, such embodiments provide advantageous improvements in accuracy of side chain torsion angle prediction in some embodiments. In such embodiments, since model training is performed on crystal data and also when the trained model is use to predict and compare with the crystal data, provision of the true crystal environment is useful to build the environmental context. Adding symmetry mates emulates the true crystal environment. In embodiments that make use of this feature, the local subgraphs that are used by the set of partial-context models and the set of full-context models described in detail below, when available, incorporate nodes and edges from atoms that belong to crystal symmetry mates along with the atoms of the default asymmetric unit of the target polymer. In such instances, the side chain dihedral angle predictions are made for the asymmetric unit only but at the end of making the prediction during each stage of prediction for the asymmetric unit, the predicted side chain dihedral angles are “mirrored” into the crystal symmetry mates using applicable crystallographic operators.

Block 232-238. Referring to block 232 of FIG. 2C, the at least one program comprises instructions for (B) sequentially inputting each first partial-context subgraph 152 in a plurality of first partial-context subgraphs of the graph 120 into a first trained partial-context graph neural network 160 thereby obtaining a plurality of first instances of calculated first side chain dihedral angles for the plurality of residues. In some such embodiments, referring block 234, the sequentially inputting (B) comprises, for each respective residue in the plurality of residues, inputting a corresponding first partial-context subgraph 152, in the plurality of first partial-context subgraphs of the graph 120, drawn from the nodes in the graph that represent backbone atoms of the respective residue or backbone atoms of the polymer proximate to the respective residue, into the first trained partial-context graph neural network, thereby obtaining a first instance of a corresponding calculated first side chain dihedral angle for the respective residue.

In some such embodiments, referring to block 236, the backbone atoms of the polymer proximate to the respective residue are a cutoff number of atoms (e.g., between 20 and 80 atoms) in the protein that are closest to the respective residue (e.g., the C carbon of the residue, the center of mass of the residue, or some other point of reference of the residue such as a designated main chain atom of the residue other than the C carbon) in the original set of M three-dimensional coordinates {x₁, . . . , x_{M}}. In alternative embodiments, the backbone atoms of the polymer proximate to the respective residue are defined as being those backbone atoms within a sphere having a predetermined radius, where the sphere is centered either on a particular atom of the identified residue (e.g., C carbon in the case of proteins) or the center of mass of the identified residue in the atomic model of the polymer. In some instances, the predetermined radius is a radius that is between 5 Angstroms and 80 Angstroms, between 10 Angstroms and 70 Angstroms, between 15 Angstroms and 65 Angstroms, or between 20 Angstroms and 60 Angstroms. For example, consider the case where the polymer is a protein comprising 200 residues and the target residue is a tyrosine at position 100 (i.e., the 100^{th} residues of the 200 residue protein). In this example, the backbone atoms that are proximate to this tyrosine is defined based on the position of the C carbon of residue 100 (or some other designated heavy atom of the residue or the center of mass of the residue) and the cutoff radius of the sphere.

In some embodiments, the first trained partial-context graph neural network 160 has 500 or more parameters, 1000 or more parameters, 10,000 or more parameters, 100,000 or more parameters or 1×10⁶ or more parameters. As used herein, the term “parameter” when used in reference to any disclosed trained partial-context graph neural network 160 or trained full-context neural network 170 refers to any coefficient or, similarly, any value of an internal or external element (e.g., a weight and/or a hyperparameter) in the model that can affect (e.g., modify, tailor, and/or adjust) one or more inputs, outputs, and/or functions in the model. For example, in some embodiments, a parameter refers to any coefficient, weight, and/or hyperparameter that can be used to control, modify, tailor, and/or adjust the behavior, learning, and/or performance of a partial-context graph neural network 160 or a full-context neural network 170. In some embodiments, a parameter is used to increase or decrease the influence of an input (e.g., a feature) to a partial-context graph neural network 160 or a full-context neural network 170. As a nonlimiting example, in some embodiments, a parameter is used to increase or decrease the influence of a node (e.g., of a neural network), where the node includes one or more activation functions. Assignment of parameters to specific inputs, outputs, and/or functions is not limited to any one paradigm for a given partial-context graph neural network 160 or full-context neural network 170 but can be used in any suitable manner for a desired performance. In some embodiments, a parameter has a fixed value. In some embodiments, a value of a parameter is manually and/or automatically adjustable. In some embodiments, a value of a parameter is modified by a validation and/or training process for a partial-context graph neural network 160 or a full-context neural network 170 (e.g., by error minimization and/or backpropagation methods). As illustrated in FIG. 1D, in some embodiments, each partial-context graph neural network 160 includes a plurality of parameters 162 and each full-context graph neural network 160 includes a plurality of parameter 172. In some embodiments, the plurality of parameters 162/172 for each such network is n parameters, where. n≥2; n≥5; n≥10; n≥25; n≥40; n 50; n≥75; n≥100; n≥125; n≥150; n≥200; n≥225; n≥250; n≥350; n≥500; n≥600; n≥750; n≥1,000; n≥2,000; n≥4,000; n≥5,000; n≥7,500; n≥10,000; n≥20,000; n≥40,000; n≥75,000; n≥100,000; n≥200,000; n≥500,000, n≥1×10⁶, n≥5×10⁶, or n≥1×10⁷. In some embodiments n is between 10,000 and 1×10⁷, between 100,000 and 5×10⁶, or between 500,000 and 1×10⁶.

Block 238. Referring to block 238, of FIG. 2C, in some embodiments, the first instance of the corresponding calculated first side chain dihedral angle for the respective residue is the X₁ side chain dihedral angle. In some embodiments, the disclosed methods only refine the protein side chain dihedral angles X₂, X₃, and X₄ and the coordinates of side chain atoms through X₁, other than C_{β}, are obtained from the initial set of M three-dimensional coordinates {x₁, . . . , x_{M}}. In such embodiments, the side chain C_{β} atoms are deterministically predicted using a conventional C_{β} residue builder tool based on the coordinates of the inputted backone atoms and canonical parameters for bond length and angles for the C_{β} atom. In such embodiments, the corresponding calculated first side chain dihedral angle for the respective residue is the X₂ side chain dihedral angle for the respective residue. In some embodiments, the disclosed methods only refine the protein side chain dihedral angles X₃ and X₄ and the coordinates of side chain atoms through X₂ are obtained from the initial set of M three-dimensional coordinates {x₁, . . . , x_{M}}. In such embodiments, the corresponding calculated first side chain dihedral angle for the respective residue is the X₃ side chain dihedral angle for the respective residue.

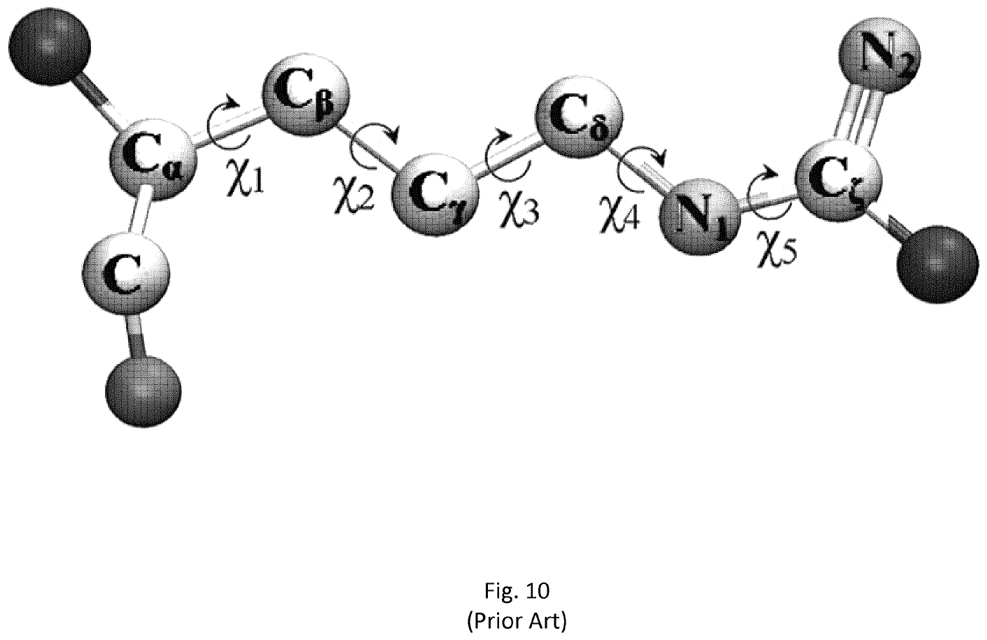

Blocks 240-242. Referring to block 240 of FIG. 2D, the at least one program comprises instructions for (C) updating the graph 120 up to the first side chain dihedral angle of each residue in the plurality of residues using the plurality of first instances of calculated first side chain dihedral angles. For instance, referring to block 242, in some embodiments the updating (C) comprises, for each respective residue in the plurality of residues, the corresponding first instance of the corresponding calculated first side chain dihedral angle is used to update the graph of the polymer to include nodes and edges for atoms of the respective residue up to the first side chain dihedral angle of the respective residue. For example, referring to FIG. 10, in the case where a residue in the plurality of residues is arginine, and the first side chain dihedral angle is X₁, the calculated dihedral angle for the arginine is used to determine the three-dimensional coordinates of the C_{γ} carbon of the arginine. The elucidated coordinates of the C_{γ} carbon of arginine, in turn, are used to update the graph 120. This will necessarily affect subgraphs drawn from the graph 120 in subsequent refinement stages such as the one discussed with reference to blocks 244-248, below.

Blocks 244-248. Referring to block 244 of FIG. 2D, the at least one program comprises instructions for (D) sequentially inputting each second partial-context subgraph in a plurality of second partial-context subgraphs of the graph 120 into a second trained partial-context graph neural network 160-2 having at least 500 parameters, thereby obtaining a plurality of first instances of calculated second side chain dihedral angles for residues in the plurality of residues. For instance, referring to block 242, in some embodiments the sequentially inputting (D) comprises, for each respective residue in the plurality of residues having a second side chain dihedral angle, inputting a corresponding second partial-context subgraph, in the plurality of second partial-context subgraphs of the graph, drawn from the nodes 121 in the graph 120 that represent backbone atoms or side chain atoms of up to the first side chain dihedral angle of (i) the respective residue or (ii) residues proximate to the respective residue, into the second trained partial-context graph neural network, thereby obtaining a first instance of a corresponding calculated second side chain dihedral angle for the respective residue. Thus, for instance, referring to FIG. 10, in the case of arginine where the corresponding calculated second side chain dihedral angle for the respective arginine is the X₂ side chain dihedral angle, the corresponding second partial-context subgraph would include node and edge representations of the main-chain atoms and the C_{β} and C_{γ} side chain atoms for the respective arginine. In some embodiments, referring to block 248, the first instance of the corresponding calculated second side chain dihedral angle for the respective residue is the X₂ side chain dihedral angle for the respective residue.

Blocks 250-252. Referring to block 250 of FIG. 2E, the at least one program comprises instructions for (E) updating the graph up to a level of a second side chain dihedral angle using the plurality of first instances of calculated second side chain dihedral angles. In some embodiments, referring to block 252, the updating (E) comprises, for each respective residue in the plurality of residues having a second side chain dihedral angle, using the corresponding first instance of the corresponding calculated second side chain dihedral angle to update the graph to include nodes and edges for atoms of the respective residue up to the second dihedral angle. For example, referring to FIG. 10, in the case where a residue in the plurality of residues is arginine, and the second side chain dihedral angle is X₂, the calculated X₂ dihedral angle for the arginine is used to determine the three-dimensional coordinates of the C_{δ} carbon of the arginine. The elucidated coordinates of the C_{δ} carbon of arginine, in turn, are used to update the graph 120. This will necessarily affect subgraphs drawn from the graph 120 in subsequent refinement stages.

Blocks 254-266. Referring to block 254 of FIG. 2E, the at least one program comprises instructions for (F) updating the graph with updated side chain dihedral angle values obtained by sequentially inputting a plurality of full-context subgraphs, each full-context subgraph in the plurality of full-context subgraphs associated with a different residue in the plurality of residues, into a plurality of trained full-context graph neural networks, each having at least 500 parameters, thereby elucidating the side chain dihedral angle values for the plurality of residues.

This represents the second phase of the disclosed systems and methods, in which a switch from partial-context to full-context is used. In more detail, in some embodiments, referring to block 258, the updating (F) comprises the following procedure.

First, referring to block 260 of FIG. 2F, (i) for each respective residue in the plurality of residues, a corresponding first full-context subgraph 152 drawn from the nodes in the graph, other than side chain atoms of the respective residue other than C_{β}, is inputted into a first trained full-context graph neural network 170-1 in the plurality of trained full-context graph neural networks, thereby obtaining a second instance of a corresponding calculated first side chain dihedral angle (e.g., X₁) for the respective residue.

Second, referring to block 262 of FIG. 2F, (ii) for each respective residue in the plurality of residues, the second instance of the corresponding calculated first side chain dihedral angle is used to update the corresponding distance relationship of edges in the graph affected by the second instance of the corresponding calculated first side chain dihedral angle. For example, referring to FIG. 10, in the case where a residue in the plurality of residues is arginine, and the first side chain dihedral angle is X₁, the second instance of the corresponding calculated first side chain dihedral angle X₁ for the arginine is used to re-determine the three-dimensional coordinates of the C_{γ} carbon of the arginine. It will be recalled that the first trained partial-context graph neural network 160-1 determined the first instance of the corresponding calculated first side chain dihedral angle X₁ for the arginine and thus the coordinates of the C_{γ} carbon of the arginine in the first instance. The re-elucidated coordinates of the C_{γ} carbon of arginine from the computation of the X₁ angle for arginine by the first trained full-context graph neural network 170-1, in turn, are used to update the graph 120. This will necessarily affect subgraphs drawn from the graph 120 in subsequent refinement stages.

Third, referring to block 264 of FIG. 2F, (iii) for each respective residue in the plurality of residues having a second side chain dihedral angle (e.g., X₂) a corresponding second full-context subgraph drawn from the nodes 121 in the graph 120, other than the nodes representing side chain atoms of the respective residue beyond the first dihedral angle (e.g., through the C_{γ} carbon for arginine), is inputted into a second trained full-context graph neural network in the plurality of trained full-context graph neural networks, thereby obtaining a second instance of a corresponding calculated second side chain dihedral angle for the respective residue.

Fourth, referring to block 266 of FIG. 2F, (iv) for each respective residue in the plurality of residues having a second side chain dihedral angle, the second instance of the corresponding calculated second side chain dihedral angle is used to update the distance relationship of each edge in the graph affected by the second instance of the corresponding calculated second side chain dihedral angle. For example, referring to FIG. 10, in the case where a residue in the plurality of residues is arginine, and the second side chain dihedral angle is X₂, the second instance of the corresponding calculated second side chain dihedral angle X₂ for the arginine is used to re-determine the three-dimensional coordinates of the C_{δ} carbon of the arginine. It will be recalled that the second trained partial-context graph neural network 160-2 determined the first instance of the corresponding calculated first side chain dihedral angle X₂ for the arginine and thus the coordinates of the C_{δ} carbon of the arginine in the first instance. The re-elucidated coordinates of the C_{δ} carbon of arginine from the computation of the X₂ angle for arginine by the second trained full-context graph neural network 170-2, in turn, are used to update the graph 120. This will necessarily affect subgraphs drawn from the graph 120 in subsequent refinement stages.

Blocks 270-272. Referring to block 270 of FIG. 2G, in some embodiments, the first trained partial-context graph neural network, the second trained partial-context graph neural network, and each trained full-context graph neural network in the plurality of trained full-context graph neural networks is a message passing graph neural network. See Gilmer et al, 2017, “Neural message passing for quantum chemistry,” In: Proceedings of the 34th International Conference on Machine Learning volume 70, JMLR. Org, pp. 1263-1272; and Maguire et al., 2021, “XENet: Using a new graph convolution to accelerate the timeline for protein design on quantum computers,” PLoS Comput Biol 17(9): e1009037, each of which is hereby incorporated by reference. In particular, message passing graph neural networks act on the node attributes of a graph according to the following general scheme:

$${x}_{i}^{′} = γ ( {x}_{i} , {□}_{( j ∈ N ( i )} ϕ ( {x}_{i} , {x}_{j} , {e}_{( j , i )} ) ) , ∀ i ∈ V$$

where ϕ is a message function that depends on the graph's nodes and edge attributes (respectively X and E), [symbol] is any permutation invariant operation that aggregates messages coming from the neighborhood of i, and γ is an update function. Further notation not referenced here is as in Maguire et al, Id. Intuitively, message-passing graph neural networks transform the attributes of the graph by exchanging information between neighboring nodes. While the equation shown above only updates the nodes through message passing, with XENet both nodes and edges and updated with message passing.

Referring to block 272, in one particular implementation, the first trained partial-context graph neural network, the second trained partial-context graph neural network, and each trained full-context graph neural network in the plurality of trained full-context graph neural networks comprises an embedding layer for receiving embedded graph information associated with a residue in the polymer, followed by a plurality of layers that each convolve over both a plurality of edge attributes and a plurality of node attributes, followed by an average pooling layer employed to the nodes corresponding to atoms in the respective residue, followed by a multi-layered perceptron with an activation function (e.g., tanh, because outputs of sine and cosine are bounded by [−1,1]) having two output channels, where the output channels give a sine and a cosine value for a side chain dihedral angle of the respective residue.

Block 276. Referring to block 276 in FIG. 2H, in some embodiments where the polymer is a protein, prior to the updating (F), for each respective residue in the plurality of residues having a X₃ side chain dihedral angle, a corresponding third partial-context subgraph drawn from the nodes in the graph that represent backbone atoms or side chain atoms up to the second side chain dihedral angle of (i) the respective residue or (ii) residues proximate to the respective residue, is inputted into a third trained partial-context graph neural network 160-3 having at least 500 parameters 162, thereby obtaining a first instance of a corresponding calculated X₃ dihedral angle for the respective residue. For each respective residue in the plurality of residues having a X₃ dihedral angle, the corresponding first instance of the corresponding calculated X₃ dihedral angle is used to update the graph to include nodes and edges for atoms of the respective residue up to the X₃ dihedral angle. For example, referring to FIG. 10, in the case where a residue in the plurality of residues is arginine, first instance of a corresponding calculated X₃ dihedral angle for the arginine is used to determine the three-dimensional coordinates of the N₁ nitrogen of the arginine. The elucidated coordinates of the N₁ nitrogen of arginine, in turn, are used to update the graph 120. This will necessarily affect subgraphs drawn from the graph 120 in subsequent refinement stages.

In such embodiments in accordance with block 276, the updating (F) further comprises (v) for each respective residue in the plurality of residues having a X₃ dihedral angle, inputting a corresponding third full-context subgraph drawn from the nodes in the graph, other than side chain atoms of the respective residue beyond the second dihedral angle, into a third trained full-context graph neural network 160-3 in the plurality of trained full-context graph neural networks, thereby obtaining a second instance of a corresponding calculated X₃ dihedral angle for the respective residue, and (vi) for each respective residue in the plurality of residues having a X₃ dihedral angle, using the second instance of the corresponding calculated X₃ dihedral angle to update the distance relationship of each edge in the graph affected by the second instance of the corresponding calculated X₃ dihedral angle. While the third trained partial-context graph neural network 160-3 determined the first instance of the corresponding calculated third side chain dihedral angle X₃ for the arginine and thus the coordinates of the N₁ nitrogen of the arginine in the first instance, the re-elucidated coordinates of the N₁ nitrogen of the arginine from the computation of the X₃ angle for arginine by the third trained full-context graph neural network 170-3, in turn, are used to re-update the graph 120 once again. This will necessarily affect subgraphs drawn from the graph 120 in subsequent refinement stages.

Block 280. Referring to block 280 of FIG. 2I, prior to the updating (F), for each respective residue in the plurality of residues having a X₄ dihedral angle, a corresponding fourth partial-context subgraph drawn from the nodes in the graph that represent backbone atoms or side chain atoms of up to the X₃ dihedral angle of (i) the respective residue or (ii) residues proximate to the respective residue, is inputted into a fourth trained partial-context graph neural network having at least 500 parameters, thereby obtaining a first instance of a corresponding calculated X₄ dihedral angle for the respective residue. For each respective residue in the plurality of residues having a X₄ dihedral angle, use the corresponding first instance of the corresponding calculated X₄ dihedral angle to update the graph to include nodes and edges for atoms of the respective residue through the X₄ dihedral angle. In such embodiments, the updating (F) further comprises: (vi) for each respective residue in the plurality of residues having a X₄ dihedral angle, inputting a corresponding fourth full-context subgraph drawn from the nodes in the graph, other than side chain atoms of the respective residue beyond the X₃ angle, into a fourth trained full-context graph neural network in the plurality of trained full-context graph neural networks, thereby obtaining a second instance of a corresponding calculated X₄ dihedral angle for the respective residue, and (vi) for each respective residue in the plurality of residues having a X₄ dihedral angle, using the second instance of the corresponding calculated X₄ dihedral angle to update the distance relationship of each edge in the graph affected by the second instance of the corresponding calculated X₄ dihedral angle.

Blocks 282-286. Referring to block 286 of FIG. 2J, in some embodiments the at least one program further comprises instructions for repeating the sequentially inputting (B), updating (C), sequentially inputting (D), updating (E), and updating (F) until a side chain dihedral angle convergence criterion is satisfied. Referring to block 286, in some embodiments, the side chain dihedral angle convergence criterion is an average change in side chain dihedral angle across the plurality of residues after repetition of the sequentially inputting (B), updating (C), sequentially inputting (D), updating (E), and updating (F) dropping below a threshold value. In some embodiments, this threshold value is that the root-mean-square deviation of the atomic positions between the coordinates of the side chains of the plurality of residues drops before and after one instance of the repetition of the inputting (B), updating (C), sequentially inputting (D), updating (E), and updating (F) is less than 0.5 Angstroms, less than 0.4 Angstroms, less than 0.3 Angstroms, less than 0.2 Angstroms, less than 0.1 Angstroms, less than 0.05 Angstroms or is zero.

In some embodiments, the side chain dihedral angle convergence criterion is satisfied when, for every respective side chain dihedral angle, for all amino acid residues within the plurality of residues, the maximum of the difference between the side chain predicted dihedral angle in the current iteration to the previous iteration is below a chosen tolerance. In such embodiments, all side chain dihedral angles for all residues in the plurality of residues must be below the chosen tolerance to satisfy the side chain dihedral angle convergence criterion. In some embodiments, the side chain dihedral angle convergence criterion is ten degrees or less, five degrees or less, four degrees or less, three degrees or less, two degrees or less, one degree or less, 0.5 degrees or less, 0.4 degrees or less, 0.3 degrees or less, 0.2 degrees or less, or 0.1 degrees or less.

Blocks 288-292. Referring to block 288 of FIG. 2J, in some embodiments, the at least one program further comprises instructions for training the first trained partial-context graph neural network, the second trained partial-context graph neural network, and each trained full-context graph neural network in the plurality of trained full-context graph neural networks using a loss function that trains unambiguous side chain dihedral angles as a regression task and ambiguous side chain dihedral angles by considering the lower of the two possible losses attributable to the ambiguous side chain dihedral angle Xᵢ. Referring to block 290, in some embodiments, the regression task a mean squared error loss function, a mean absolute error loss function, a Huber loss function, a Log-Cosh loss function, or a quantile loss function. See Wang et al., 2020, “A Comprehensive Survey of Loss Functions in Machine Learning,” Annals of Data Science, https://doi.org/10.1007/s40745-020-00253-5, last accessed Sep. 15, 2021, which is hereby incorporated by reference. Referring to block 292, in some embodiments a first loss in the two possible losses is for a side chain dihedral angle value for X₄ and the second loss in the two possible losses is a for a side chain dihedral angle value for Xᵢ−π. In some embodiments, periodicity in [−π, π] is taken into account while calculating the loss and also when computing the minimum of the loss pertaining to Xᵢ and Xᵢ−π.

Block 294-302. Referring to block 294 of FIG. 2K, in some embodiments, the at least one program further comprises instructions for using the elucidated side chain dihedral angle values for the plurality of residues to determine an interaction score between the polymer and a composition.

For example, referring to block 298 of FIG. 2K, in some embodiments the polymer is an enzyme, the composition is being screened in silico to assess an ability to inhibit an activity of the enzyme, and the interaction score is a calculated binding coefficient, IC₅₀, EC₅₀, Kd, KI, or pKI of the composition to the first enzyme. Measured binding coefficients, IC₅₀, EC₅₀, Kd, KI, and pKI are generally described in Huser ed., 2006, High-Throughput-Screening in Drug Discovery, Methods and Principles in Medicinal Chemistry 35; and Chen ed., 2019, A Practical Guide to Assay Development and High-Throughput Screening in Drug Discovery, each of which is hereby incorporated by reference. In some embodiments, the composition satisfies any two or more rules, any three or more rules, or all four rules of the Lipinski's rule of Five: (i) not more than five hydrogen bond donors, (ii) not more than ten hydrogen bond acceptors, (iii) a molecular weight under 500 Daltons, and (iv) a Log P under 5. See, Lipinski, 1997, Adv. Drug Del. Rev. 23, 3, which is hereby incorporated herein by reference in its entirety. In some embodiments, the composition satisfies one or more criteria in addition to Lipinski's Rule of Five. For example, in some embodiments, the composition has five or fewer aromatic rings, four or fewer aromatic rings, three or fewer aromatic rings, or two or fewer aromatic rings. In some embodiments, the composition is any organic compound having a molecular weight of less than 2000 Daltons, of less than 4000 Daltons, of less than 6000 Daltons, of less than 8000 Daltons, of less than 10000 Daltons, or less than 20000 Daltons.

As another example, referring to block 300 of FIG. 2K, in some embodiments the polymer is a first protein, the composition is a second protein being screened in silico to assess an ability to bind to the first protein in order to inhibit or enhance an activity of the first protein, and the interaction score is a calculated binding coefficient of the second protein to the first protein.

As still another example, referring to block 302 of FIG. 2K, in some embodiments the polymer is a first Fc fragment of a first type, the composition is a second protein is Fc fragment of a second type, and the interaction score is a calculated binding coefficient of the second Fc fragment to the first Fc fragment.

In some embodiments any of the methods disclosed herein make use of the interaction score of the composition to develop a treatment of a medical condition associated with the polymer. In some such embodiments, the treatment comprises the composition and one or more excipients and/or pharmaceutically acceptable carrier and/or dileuent. These include all conventional solvents, dispersion media, fillers, solid carriers, coatings, antifungal and antibacterial agents, dermal penetration agents, surfactants, isotonic and absorption agents and the like. It will be understood that the compositions of the invention may also include other supplementary physiologically active agents.

An exemplary carrier is pharmaceutically “acceptable” in the sense of being compatible with the other ingredients of the composition and not injurious to the patient. The compositions may conveniently be presented in unit dosage form and may be prepared by any methods well known in the art of pharmacy. Such methods include the step of bringing into association the active ingredient with the carrier that constitutes one or more accessory ingredients. In general, the compositions are prepared by uniformly and intimately bringing into association the active ingredient with liquid carriers or finely divided solid carriers or both, and then if necessary shaping the product.

Exemplary compounds, compositions or combinations of the invention formulated for intravenous, intramuscular or intraperitoneal administration, and a compound of the invention or a pharmaceutically acceptable salt, solvate or prodrug thereof may be administered by injection or infusion.

Injectables for such use can be prepared in conventional forms, either as a liquid solution or suspension or in a solid form suitable for preparation as a solution or suspension in a liquid prior to injection, or as an emulsion. Carriers can include, for example, water, saline (e.g., normal saline (NS), phosphate-buffered saline (PBS), balanced saline solution (BSS)), sodium lactate Ringer's solution, dextrose, glycerol, ethanol, and the like; and if desired, minor amounts of auxiliary substances, such as wetting or emulsifying agents, buffers, and the like can be added. Proper fluidity can be maintained, for example, by using a coating such as lecithin, by maintaining the required particle size in the case of dispersion and by using surfactants.

The compound, composition or combinations of the invention may also be suitable for oral administration and may be presented as discrete units such as capsules, sachets or tablets each containing a predetermined amount of the active ingredient; as a powder or granules; as a solution or a suspension in an aqueous or non-aqueous liquid; or as an oil-in-water liquid emulsion or a water-in-oil liquid emulsion. The active ingredient may also be presented as a bolus, electuary or paste. In another embodiment, the compound of formula (I) or a pharmaceutically acceptable salt, solvate or prodrug is orally administerable.

A tablet may be made by compression or moulding, optionally with one or more accessory ingredients. Compressed tablets may be prepared by compressing in a suitable machine the active ingredient in a free-flowing form such as a powder or granules, optionally mixed with a binder (e.g inert diluent, preservative disintegrant (e.g. sodium starch glycolate, cross-linked polyvinyl pyrrolidone, cross-linked sodium carboxymethyl cellulose) surface-active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein using, for example, hydroxypropylmethyl cellulose in varying proportions to provide the desired release profile. Tablets may optionally be provided with an enteric coating, to provide release in parts of the gut other than the stomach.

The compound, composition or combinations of the invention may be suitable for topical administration in the mouth including lozenges comprising the active ingredient in a flavored base, usually sucrose and acacia or tragacanth gum; pastilles comprising the active ingredient in an inert basis such as gelatine and glycerin, or sucrose and acacia gum; and mouthwashes comprising the active ingredient in a suitable liquid carrier.

The compound, composition or combinations of the invention may be suitable for topical administration to the skin may comprise the compounds dissolved or suspended in any suitable carrier or base and may be in the form of lotions, gel, creams, pastes, ointments and the like. Suitable carriers include mineral oil, propylene glycol, polyoxyethylene, polyoxypropylene, emulsifying wax, sorbitan monostearate, polysorbate 60, cetyl esters wax, cetearyl alcohol, 2-octyldodecanol, benzyl alcohol and water. Transdermal patches may also be used to administer the compounds of the invention.

The compound, composition or combination of the invention may be suitable for parenteral administration include aqueous and non-aqueous isotonic sterile injection solutions which may contain anti-oxidants, buffers, bactericides and solutes which render the compound, composition or combination isotonic with the blood of the intended recipient; and aqueous and non-aqueous sterile suspensions which may include suspending agents and thickening agents. The compound, composition or combination may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

It should be understood that in addition to the active ingredients particularly mentioned above, the composition or combination of this invention may include other agents conventional in the art having regard to the type of composition or combination in question, for example, those suitable for oral administration may include such further agents as binders, sweeteners, thickeners, flavouring agents disintegrating agents, coating agents, preservatives, lubricants and/or time delay agents. Suitable sweeteners include sucrose, lactose, glucose, aspartame or saccharine. Suitable disintegrating agents include cornstarch, methylcellulose, polyvinylpyrrolidone, xanthan gum, bentonite, alginic acid or agar. Suitable flavouring agents include peppermint oil, oil of wintergreen, cherry, orange or raspberry flavouring. Suitable coating agents include polymers or copolymers of acrylic acid and/or methacrylic acid and/or their esters, waxes, fatty alcohols, zein, shellac or gluten. Suitable preservatives include sodium benzoate, vitamin E, alpha-tocopherol, ascorbic acid, methyl paraben, propyl paraben or sodium bisulphite. Suitable lubricants include magnesium stearate, stearic acid, sodium oleate, sodium chloride or talc. Suitable time delay agents include glyceryl monostearate or glyceryl distearate.

In some embodiments, the medical condition is inflammation or pain. In some embodiments the medical condition is a disease. In some embodiments, the medical condition is asthma, an autoimmune disease, autoimmune lymphoproliferative syndrome (ALPS), cholera, a viral infection, Dengue fever, an E. coli infection, Eczema, hepatitis, Leprosy, Lyme Disease, Malaria, Monkeypox, Pertussis, a Yersiniapestis infection, primary immune deficiency disease, prion disease, a respiratory syncytial virus infection, Schistosomiasis, gonorrhea, genital herpes, a human papillomavirus infection, chlamydia, syphilis, Shigellosis, Smallpox, STAT3 dominant-negative disease, tuberculosis, a West Nile viral infection, or a Zika viral infection. In some embodiments, the medical condition is a disease references in Lippincott, Williams & Wilkins, 2009, Professional Guide to Diseases, 9^{th} Edition, Wolters Kluwere, Philadelphia, Pennsylvania, which is hereby incorporated by reference.

Block 304. Referring to block 304 of FIG. 2K, in some embodiments, the polymer is a protein with one or more mutations (e.g., point mutations) introduced into the protein and the at least one program further comprises instructions for using the elucidated side chain dihedral angle values for the plurality of residues to determine an effect of the one or more mutations on an activity of the protein relative to an activity of a wild-type naturally occurring version of the protein.

### Example 1—Model Training

For training, four partial-context models (160-1, 160-2, 160-3, and 160-4) and four full-context models (170-1, 170-2, 170-3, and 170-4) were constructed. Each constructed model had an embedding layer for receiving embedded graph information associated with a residue in a polymer, followed by a plurality (e.g., two) of layers that each convolve over both a plurality of edge attributes and a plurality of node attributes (see, for example, the XENet layer disclosed in Maquire et al., 2021, “XENet: Using a new graph convolution to accelerate the timeline for protein design on quantum computers,” PLoS Comput Biol 17(9): e1009037, which is hereby incorporated by reference), followed by an average pooling layer employed to the nodes corresponding to atoms in the respective residue, followed by a multi-layered perceptron with an activation function (e.g., tanh activation) having two output channels, where the output channels give a sine and a cosine value for a side chain dihedral angle of the respective residue. Mean squared error (MSE) was used as the loss function for training. This error was computed using the output of the neural network using the sine and cosine of the side chain dihedral angle, Xᵢ, to account for periodicity.

A total of 4000 high resolution PDB structures with a maximum of 300 residues without any chain breaks due to missing residues were used for training the four partial-context and four-full context models. A total of 1000 structures were used as the validation set. Alternate conformations were removed from the PDB entries used for training. Subgraphs for residues with missing side chain or side chains with low electron density were omitted. In particular, RSRZ outliers detected using the REST Api, https://www.ebi.ac.uk/pdbe/api/doc, last accessed Oct. 19, 2021, which is hereby incorporated by reference, were omitted.

Ambiguity in the flip state of asparagine and histidine X₂ and glutamine X₃ side chain dihedral angles was addressed using Amber's reduce —FLIPs. The ambiguity in prediction stemming from structural symmetry in X₂ of aspartic acid, phenylalanine, and tyrosine and X₂ of glutamic acid was treated as special case in the loss function calculations where the smaller loss of the two pertaining to Xᵢ and Xᵢ−π was used.

The computational framework was adapted to handle structures with and without crystal symmetry mates. In the case of structures with crystal symmetry mates only the asymmetric unit (AU) was used to compute the loss function although the input graph attributes comprised of atoms from AU's and their symmetry mates. The final model trained for evaluation were ones generated using the crystal symmetry mate information.

As the trained models were obtained by training directly on PDB data, they learned higher order correlation between the graph nodes that are within the receptive field, e.g., the environmental context via a set of geometric features embedded into the edge attributes and therefore managed to quickly self-evolve and rectify their state till convergence within a few (˜5) iteration cycles.

### Example 2—Side Chain Prediction

The established, standard state-of-the-art packing algorithms attempt to solve the combinatorial optimization problem, which arises when the conformation of multiple side chains in a protein are being predicted, to find the best set of discrete rotamer conformations sample from pre-computed rotamer libraries that minimizes predefined empirically tuned energy/scoring functions. One embodiments of the present disclosure, ZymePackNet, provides a novel two stage computational framework, that uses a set of regression models trained on high resolution, non-redundant PDB crystal structures as described in Examiner 1. ZymePackNet thereby circumvents issues beleaguered by the choice of rotamer libraries or empirical scoring metrics as elaborated above. An advantage of ZymePackNet lies in the novelty of the computational framework that allows for the deterministic prediction and further refinement of the side chain conformation iteratively conditioned upon the previous state without sifting through the rotamer libraries, requiring an energy function, or without having to rely on methods like dead end elimination or other sampling techniques unlike other packing tools.

The first stage of the side chain prediction involves generating an initial set of side chains starting from the protein backbone using a set of trained models (PC models 160-1, 160-2, 160-3, and 160-4) that utilizes and predicts the side chain dihedral angles hierarchically from X₁ to X₄ conditioned upon the amount of information available at the time of prediction. In more detail, to predict the side chain conformations starting from only the protein backbone, the trained PC-X₁ model 160-1 was first employed on the graph generated using only backbone atoms. To apply PC-X₁ to build the side chain through the X₁ side chain dihedral angle, the coordinates of the C_{β} atom were needed in order to predict the side chain dihedral angle X₁ of the target residue. Given a protein backbone, the side chain C_{β} atom in the target residue side chain was first deterministically predicted using in an house conventional C_{β} residue builder tool based on the coordinates of the backbone atoms of the polymer. Once the coordinates of the C_{β} atom for the target residue were populated, they were used to include the C_{β} atom subgraph for the target residue. Further, the trained PC-X₁ model 160-1 model was applied on the updated subgraph generated using backbone atoms and the side chain C_{β} atom. Once X₁ was predicted for each of the residues in the proteins, all the atoms were populated up to the level of X₁ and the PC-X₂ (160-2) through PC-X₄ (160-4) models were employed in a similar fashion conditioned upon the graph generated in the previous prediction.

The second stage was an iterative self-distillation stage, where the protein graph self corrects itself conditioned upon the neighbourhood aggregation scheme upon the previous state of the graph, using another set of trained models (FC models 170-1, 170-2, 170-3, and 170-4). Thus, the final output structure of the PC model 160-4 containing full side chain description was refined using an iterative refinement cycle using the set of FC models 170-1, 170-2, 170-3, and 170-4. During each iteration, FC-X₁ (170-1) through FC-X₄ (170-4), FC models were employed sequentially followed by updating the graph for the whole structure. At the end of each iteration, the prediction of the dihedral angles was compared with the previous round the iteration through the four PC models and the four FC models was stopped when the change in prediction reached a desired tolerance. The rationale for using the proposed two stage prediction framework was to first populate the sidechains to a satisfactory conformation using the PC models which was further conditionally refined with more context via FC models such that fewer iterations were required for convergence compared to the alternate of using random initial conformations for the side chains as starting structure for the FC refinements. The PC stage (without the FC refinement stage), although providing satisfactory results, resulted in poorer accuracy than the disclosed two stage refinement framework.

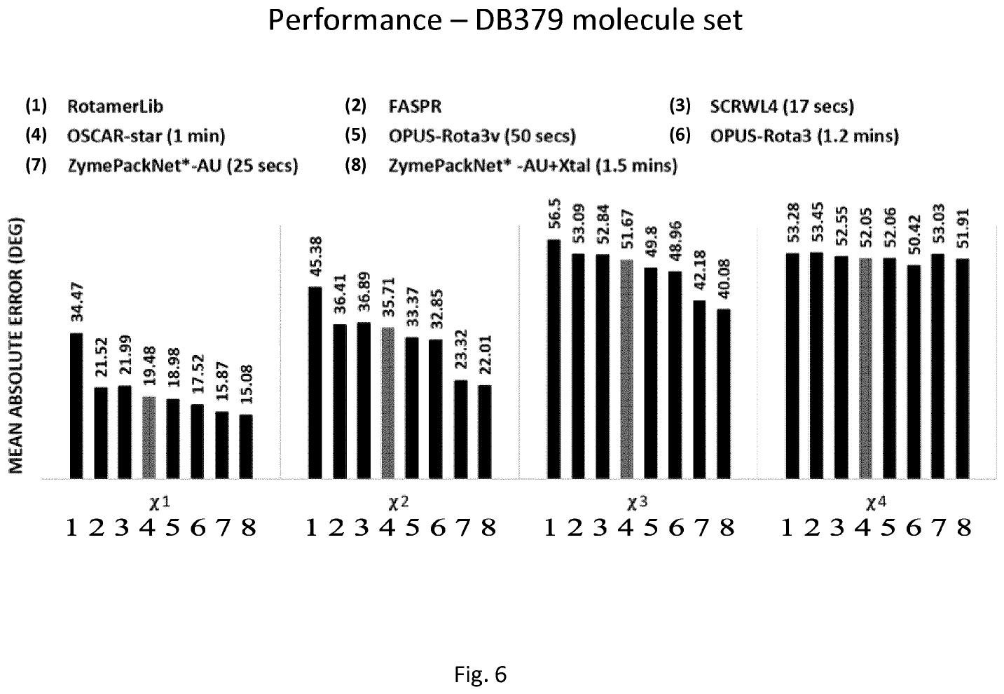

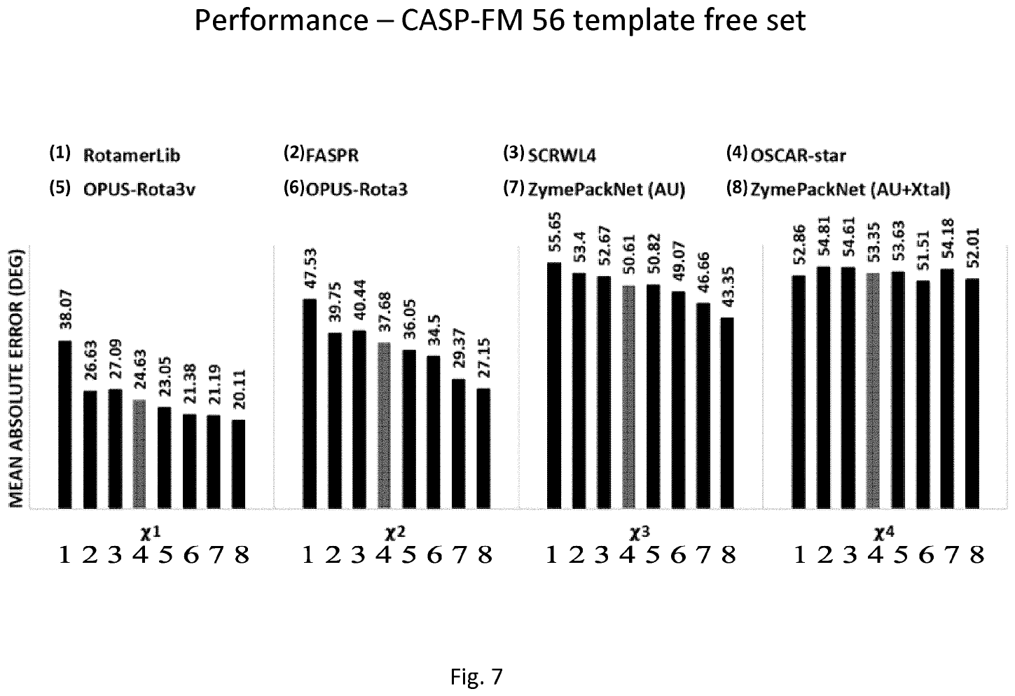

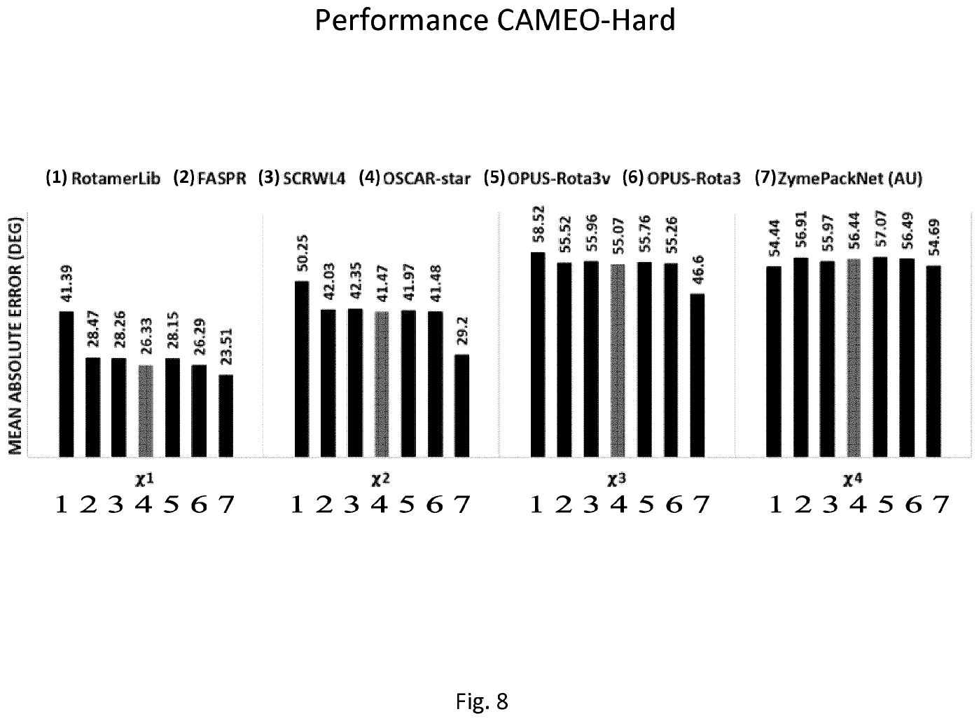

With reference to FIGS. 6, 7, and 8, using the respective datasets DB379 (See, Krivov et al., 2009, “Improved prediction of protein side-chain conformations with SCWRL4.” Proteins, 77, 778-795, which is hereby incorporated by reference), CASP-FM 56, containing 56 template-free modeling (FM) proteins collected from CASP10 to CASP13 (See, Uddin et al., 2020, “Self-Attention Augmented Inception-Inside-Inception Network Improves Protein Secondary Structure Prediction,” Bioinformatics 4599, which is hereby incorporated by reference), and CAMEO-Hard, which contain 61 proteins labeled as hard targets (See, Haas et al., 2018, “Continuous Automated Model EvaluatiOn (CAMEO) complementing the critical assessment of structure prediction in CASP12,” Proteins, 86, pp. 387-398, which is hereby incorporated by reference), the accuracy of the disclosed algorithm, embodied as both ZymePackNet—AU (asymmetric unit only) and ZymePackNet —AU+Xtal (including symmetry mates), was benchmarked against the conventional side chain packing tools RotamerLib (Shapovalov and Dunbrack, 2011, “A smoothed backbone dependent rotamer library for proteins derived from adaptive kernel density estimates and regressions,” Structure 19, pp. 844-858), Oscar-star (Liang et al., 2011 “Fast and accurate prediction of protein side-chain conformations,” Bioinformatics 27, pp. 2913-2914), FASPR (Huang et al., 2020, “FASPR: an open-source tool for fast and accurate protein side-chain packing,” Bioinformatics 36, pp. 3758-3765.), OPUS-Rota3v (Xu et al., 2020, “Opus-Rota3: Improving Side-Chain Modeling by Deep Neural Networks and Ensemble Methods,” J. Chem. Inf. Model. 60, pp. 6691-6697), SCRWL4 (Krivov et al., 2009 “Jr. Improved prediction of protein side-chain conformations with SCWRL4,” Proteins, 77, pp. 778-795), and OPUS-Rota3 (Xu et al., 2020, “Opus-Rota3: Improving Side-Chain Modeling by Deep Neural Networks and Ensemble Methods,” J. Chem. Inf. Model 60, pp. 6691-6697). Accuracy was benchmarked in units of mean absolute error between predicted side chain dihedral angles and actual side chain dihedral angles across the three datasets.

As illustrated in FIGS. 6, 7, and 8, for all three datasets, both ZymePackNet—AU+Xtal and ZymePackNet—AU outperformed in accuracy (measured as mean absolute error with respect to the PDB structures in the DB389 dataset) approximately by 2°, 10°, and 6° for X₁, X₂, and X₃ respectively while for X₄ accuracy was slightly worse (˜1) than only one model, OPUS-Rota3 when crystal symmetry mates were not considered (ZympePackNet—AU). When crystal symmetry mates were considered, ZymePackNet outperformed all examined methods and improved predictions by ˜1° for all side chain dihedrals compared to ZymePackNet without crystal symmetry mates considered for predictions. Note that crystal information was missing for the Cameo-Hard dataset and so only ZymePackNet (AU) was run against the CAMEO-Hard dataset.

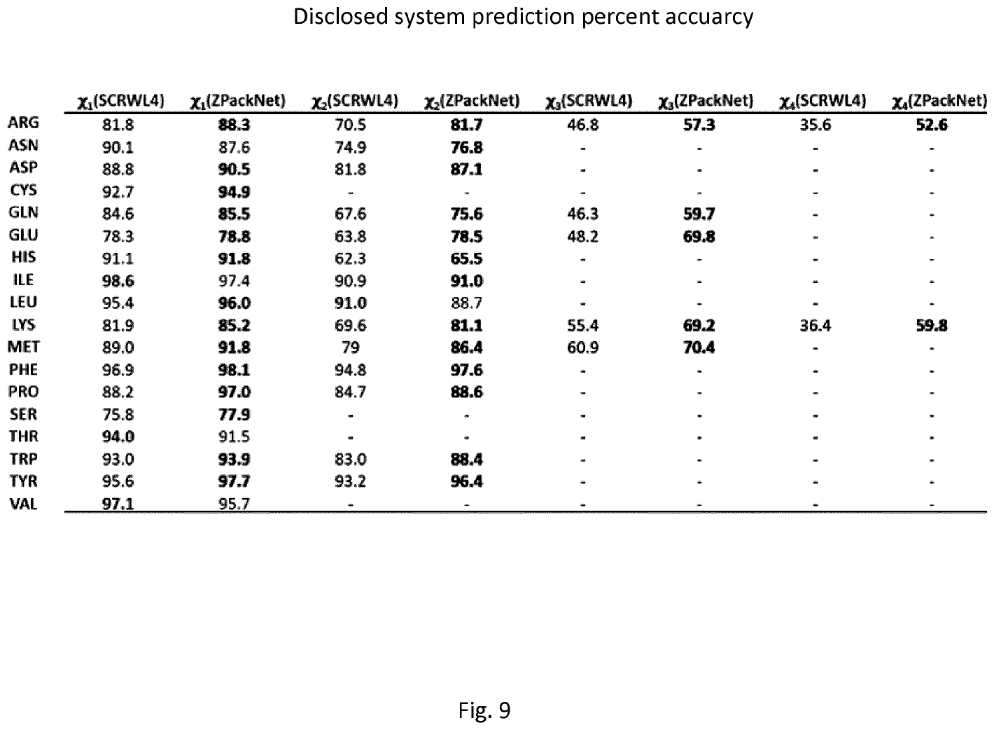

Although not illustrated in FIGS. 6, 7, and 8, when compared against a ZymePack, which is used for side chain packing and is algorithmically similar to SCRWL4 that uses a backbone independent rotamer library and in-house scoring functions, significant gain in computational efficiency was observed by ZymePackNet (AU) and ZymePackNet (AU+Xtal). The average run time per structure across the DB379, CASP-FM 56, and CAMEO-Hard datasets using ZymePack is 4-6 hours whereas the average runtime of ZymePackNet is 23 secs per structure. As illustrated in FIG. 9 approximately 5-10° improvement in prediction accuracy for all dihedrals angles were seen for the disclosed side chain packing method (denoted ZPackNet in FIG. 9) compared to ZymePack (denoted SCRWL4 in FIG. 9) across the DB379, CASP-FM 56, and CAMEO-Hard datasets.

Although most accurate among the methods evaluated here, ZymePackNet (the side chain packing algorithm of the present disclosure that works in accordance with FIG. 2) is slower than some of the rotamer packing methods known for their speed such as FASPR and SCRWL4. For example, ZymePackNet (˜23 secs/structure without symmetry mates and ˜72 secs with symmetry mates) was substantially faster than the most accurate method OPUS-Rota3 but was about twice as slow as SCRWL4 (12 secs/structure) without symmetry mates and 3.5× slower (21 secs/structure) with the symmetry mates. However, the version of ZymePackNet run in this example recomputes the entire graph whenever the coordinates of any atom within the structure are updated during the multiple steps of the iterative refinement. Since most of the polymer and therefore the graph 120 does not change between iterations, improvement in efficiency can be realized by updating the relevant attributes of the graph state without having to recompute all attributes. Another gain in efficiency can be achieved by selectively updating the graph 120 based on the confidence of the predicted output. This can be accomplished, for example, by training an ensemble of models trained on different samples of the training data. If the models agree on a given prediction, that is taken as a high confidence prediction, and if the models disagree, then it is low confidence.

### CONCLUSION

The methods illustrated in FIG. 2 may be governed by instructions that are stored in a computer readable storage medium and that are executed by at least one processor of at least one server. Each of the operations shown in FIG. 2 may correspond to instructions stored in a non-transitory computer memory or computer readable storage medium. In various implementations, the non-transitory computer readable storage medium includes a magnetic or optical disk storage device, solid state storage devices such as Flash memory, or other non-volatile memory device or devices. The computer readable instructions stored on the non-transitory computer readable storage medium may be in source code, assembly language code, object code, or other instruction format that is interpreted and/or executable by one or more processors.

Plural instances may be provided for components, operations or structures described herein as a single instance. Finally, boundaries between various components, operations, and data stores are somewhat arbitrary, and particular operations are illustrated in the context of specific illustrative configurations. Other allocations of functionality are envisioned and may fall within the scope of the implementation(s). In general, structures and functionality presented as separate components in the exemplary configurations may be implemented as a combined structure or component. Similarly, structures and functionality presented as a single component may be implemented as separate components. These and other variations, modifications, additions, and improvements fall within the scope of the implementation(s).

It will also be understood that, although the terms “first,” “second,” etc. may be used herein to describe various elements, these elements should not be limited by these terms. These terms are only used to distinguish one element from another. For example, a first contact could be termed a second contact, and, similarly, a second contact could be termed a first contact, which changing the meaning of the description, so long as all occurrences of the “first contact” are renamed consistently and all occurrences of the second contact are renamed consistently. The first contact and the second contact are both contacts, but they are not the same contact.

The terminology used herein is for the purpose of describing particular implementations only and is not intended to be limiting of the claims. As used in the description of the implementations and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in accordance with a determination” or “in response to detecting,” that a stated condition precedent is true, depending on the context. Similarly, the phrase “if it is determined (that a stated condition precedent is true)” or “if (a stated condition precedent is true)” or “when (a stated condition precedent is true)” may be construed to mean “upon determining” or “in response to determining” or “in accordance with a determination” or “upon detecting” or “in response to detecting” that the stated condition precedent is true, depending on the context.

The foregoing description included example systems, methods, techniques, instruction sequences, and computing machine program products that embody illustrative implementations. For purposes of explanation, numerous specific details were set forth in order to provide an understanding of various implementations of the inventive subject matter. It will be evident, however, to those skilled in the art that implementations of the inventive subject matter may be practiced without these specific details. In general, well-known instruction instances, protocols, structures and techniques have not been shown in detail.

The foregoing description, for purpose of explanation, has been described with reference to specific implementations. However, the illustrative discussions above are not intended to be exhaustive or to limit the implementations to the precise forms disclosed. Many modifications and variations are possible in view of the above teachings. The implementations were chosen and described in order to best explain the principles and their practical applications, to thereby enable others skilled in the art to best utilize the implementations and various implementations with various modifications as are suited to the particular use contemplated.

What is claimed is:

1. A computer system for molecular modeling, the computer system comprising:
one or more processors; and
memory addressable by the one or more processors, the memory storing at least one program for execution by the one or more processors, wherein the at least one program comprises instructions for:
(A) obtaining a graph of at least a portion of a polymer, wherein the graph comprises a plurality of nodes and a plurality of edges, each node in the plurality of nodes representing a main chain atom of the polymer, and each respective edge in the plurality of edges encodes at least (i) a corresponding distance relationship between a corresponding pair of nodes in the plurality of nodes and (ii) a binary indicator that indicates whether or not the corresponding pair of nodes represents a pair of atoms covalently bound to each other in the polymer, and wherein the portion of a polymer comprises a plurality of residues, at least two of which have one or more side chain dihedral angles in a set of side chain dihedral angles;
(B) sequentially inputting each first partial-context subgraph in a plurality of first partial-context subgraphs of the graph into a first trained partial-context graph neural network having at least 500 parameters, thereby obtaining a plurality of first instances of calculated first side chain dihedral angles for the plurality of residues;
(C) updating the graph up to the first side chain dihedral angle of each residue in the plurality of residues using the plurality of first instances of calculated first side chain dihedral angles;
(D) sequentially inputting each second partial-context subgraph in a plurality of second partial-context subgraphs of the graph into a second trained partial-context graph neural network having at least 500 parameters, thereby obtaining a plurality of first instances of calculated second side chain dihedral angles for residues in the plurality of residues;
(E) updating the graph up to a level of a second side chain dihedral angle using the plurality of first instances of calculated second side chain dihedral angles; and
(F) updating the graph with updated side chain dihedral angle values obtained by sequentially inputting a plurality of full-context subgraphs, each full-context subgraph in the plurality of full-context subgraphs associated with a different residue in the plurality of residues, into a plurality of trained full-context graph neural networks, each having at least 500 parameters, thereby elucidating the side chain dihedral angle values for the plurality of residues.

2. The computer system of claim 1, wherein the sequentially inputting (B) comprises, for each respective residue in the plurality of residues, inputting a corresponding first partial-context subgraph, in the plurality of first partial-context subgraphs of the graph, drawn from the nodes in the graph that represent atoms of the respective residue or atoms of the polymer proximate to the respective residue, into the first trained partial-context graph neural network, thereby obtaining a first instance of a corresponding calculated first side chain dihedral angle for the respective residue.

3. The computer system of claim 2, wherein the updating (C) comprises, for each respective residue in the plurality of residues, using the corresponding first instance of the corresponding calculated first side chain dihedral angle to update the graph of the polymer to include nodes and edges for atoms of the respective residue up to the first side chain dihedral angle of the respective residue.

4. The computer system of any one of claims 1-3, wherein the sequentially inputting (D) comprises, for each respective residue in the plurality of residues having a second side chain dihedral angle, inputting a corresponding second partial-context subgraph, in the plurality of second partial-context subgraphs of the graph, drawn from the nodes in the graph that represent backbone atoms or side chain atoms of up to the first side chain dihedral angle of (i) the respective residue or (ii) residues proximate to the respective residue, into the second trained partial-context graph neural network, thereby obtaining a first instance of a corresponding calculated second side chain dihedral angle for the respective residue.

5. The computer system of claim 4, wherein the updating (E) comprises, for each respective residue in the plurality of residues having a second side chain dihedral angle, using the corresponding first instance of the corresponding calculated second side chain dihedral angle to update the graph to include nodes and edges for atoms of the respective residue up to the second dihedral angle.

6. The computer system of any one of claims 1-5, wherein the updating (F) comprises:
(i) for each respective residue in the plurality of residues, inputting a corresponding first full-context subgraph drawn from the nodes in the graph, other than side chain atoms beyond the C_{β} carbon of the respective residue, into a first trained full-context graph neural network in the plurality of trained full-context graph neural networks, thereby obtaining a second instance of a corresponding calculated first side chain dihedral angle for the respective residue,
(ii) for each respective residue in the plurality of residues, using the second instance of the corresponding calculated first side chain dihedral angle to update the corresponding distance relationship of edges in the graph affected by the second instance of the corresponding calculated first side chain dihedral angle,
(iii) for each respective residue in the plurality of residues having a second side chain dihedral angle, inputting a corresponding second full-context subgraph drawn from the nodes in the graph, other than side chain atoms of the respective residue beyond the first dihedral angle, into a second trained full-context graph neural network in the plurality of trained full-context graph neural networks, thereby obtaining a second instance of a corresponding calculated second side chain dihedral angle for the respective residue, and
(iv) for each respective residue in the plurality of residues having a second side chain dihedral angle, using the second instance of the corresponding calculated second side chain dihedral angle to update the distance relationship of each edge in the graph affected by the second instance of the corresponding calculated second side chain dihedral angle.

7. The computer system of any one of claims 1-6, wherein the corresponding distance relationship between a corresponding pair of nodes i and j in the plurality of nodes is of the form e^{-r}^{ᵢⱼ}^{²}^{/κ}, wherein
rᵢⱼ is a distance between three-dimensional coordinates for node i and three-dimensional coordinates for node j, and
κ is a square of a cuttoff distance.

8. The computer system of claim 7, wherein rᵢⱼ is in units of Å and κ is 100 Å².

9. The computer system of any one of claims 1-8, wherein each respective edge in the plurality of edges further encodes a directional feature between a corresponding pair of nodes.

10. The computer system of claim 9, wherein each respective node in the corresponding pair of nodes i, j is assigned its own local three-dimensional reference frame and the directional feature is encoded as a 1×3 additional features representing a projection of the three dimensional coordinates of the node i onto to the local three-dimensional reference frame of node j in an edge eᵢⱼ and 1×3 additional features representing a projection of the three dimensional coordinates of the node j onto to the local three-dimensional reference frame of the node i in an edge eⱼᵢ.

11. The computer system of any one of claims 1-10, wherein the polymer is a polypeptide.

12. The computer system of any one of claims 1-10, wherein the polymer is an antigen-antibody complex.

13. The computer system of any one of claims 1-12, wherein the plurality of residues comprises 50 or more residues.

14. The computer system of claim 2, wherein the first instance of the corresponding calculated first side chain dihedral angle for the respective residue is the X₁ side chain dihedral angle for the respective residue.

15. The computer system of claim 14, wherein the first instance of the corresponding calculated second side chain dihedral angle for the respective residue is the X₂ side chain dihedral angle for the respective residue.

16. The computer system of claim 15, wherein the at least one program further comprises instructions for:
prior to the updating (F), for each respective residue in the plurality of residues having a X₃ dihedral angle, inputting a corresponding third partial-context subgraph drawn from the nodes in the graph that represent backbone atoms or side chain atoms of up to the second side chain dihedral angle of (i) the respective residue or (ii) residues proximate to the respective residue, into a third trained partial-context graph neural network having at least 500 parameters, thereby obtaining a first instance of a corresponding calculated X₃ dihedral angle for the respective residue,
for each respective residue in the plurality of residues having a X₃ dihedral angle, using the corresponding first instance of the corresponding calculated X₃ dihedral angle to update the graph to include nodes and edges for atoms of the respective residue up to the X₃ dihedral angle, and
the updating (F) further comprises:
(v) for each respective residue in the plurality of residues having a X₃ dihedral angle, inputting a corresponding third full-context subgraph drawn from the nodes in the graph, other than side chain atoms of the respective residue beyond the second dihedral angle, into a third trained full-context graph neural network in the plurality of trained full-context graph neural networks, thereby obtaining a second instance of a corresponding calculated X₃ dihedral angle for the respective residue, and
(vi) for each respective residue in the plurality of residues having a X₃ dihedral angle, using the second instance of the corresponding calculated X₃ dihedral angle to update the distance relationship of each edge in the graph affected by the second instance of the corresponding calculated X₃ dihedral angle.

17. The computer system of claim 15, wherein the at least one program further comprises instructions for:
prior to the updating (F), for each respective residue in the plurality of residues having a X₄ dihedral angle, inputting a corresponding fourth partial-context subgraph drawn from the nodes in the graph that represent backbone atoms or side chain atoms of up to the X₃ dihedral angle of (i) the respective residue or (ii) residues proximate to the respective residue, into a fourth trained partial-context graph neural network having at least 500 parameters, thereby obtaining a first instance of a corresponding calculated X₄ dihedral angle for the respective residue,
for each respective residue in the plurality of residues having a X₄ dihedral angle, using the corresponding first instance of the corresponding calculated X₄ dihedral angle to update the graph to include nodes and edges for atoms of the respective residue through the X₄ dihedral angle, and
the updating (F) further comprises:
(vi) for each respective residue in the plurality of residues having a X₄ dihedral angle, inputting a corresponding fourth full-context subgraph drawn from the nodes in the graph, other than side chain atoms of the respective residue beyond the X₃ dihedral angle, into a fourth trained full-context graph neural network in the plurality of trained full-context graph neural networks, thereby obtaining a second instance of a corresponding calculated X₄ dihedral angle for the respective residue, and
(vi) for each respective residue in the plurality of residues having a X₄ dihedral angle, using the second instance of the corresponding calculated X₄ dihedral angle to update the distance relationship of each edge in the graph affected by the second instance of the corresponding calculated X₄ dihedral angle.

18. The computer system of claim 2, wherein the backbone atoms of the polymer proximate to the respective residue are a cutoff number of atoms in the polymer that are closest to the respective residue.

19. The computer system of claim 18, wherein the cutoff number of atoms is between 20 and 80 atoms.

20. The computer system of any one of claims 1-19, wherein the first trained partial-context graph neural network, the second trained partial-context graph neural network, and each trained full-context graph neural network in the plurality of trained full-context graph neural networks is a message passing graph neural network.

21. The computer system of claim 20, wherein the first trained partial-context graph neural network, the second trained partial-context graph neural network, and each trained full-context graph neural network in the plurality of trained full-context graph neural networks comprises an embedding layer for receiving embedded graph information associated with a residue in the polymer, followed by a plurality of layers that each convolve over both a plurality of edge attributes and a plurality of node attributes, followed by an average pooling layer employed to the nodes corresponding to atoms in the respective residue, followed by a multi-layered perceptron with an activation function having two output channels, wherein the output channels give a sine and a cosine value for a side chain dihedral angle of the respective residue.

22. The computer system of claim 21, wherein the activation function is tanh.

23. The computer system of any one of claims 1 through 22, wherein the at least one program further comprises instructions for:
repeating the sequentially inputting (B), updating (C), sequentially inputting (D), updating (E), and updating (F) until a side chain dihedral angle convergence criterion is satisfied.

24. The computer system of claim 23, wherein the side chain dihedral angle convergence criterion is an average change in side chain dihedral angle across the plurality of residues after repetition of the sequentially inputting (B), updating (C), sequentially inputting (D), updating (E), and updating (F) dropping below a threshold value.

25. The computer system of any one of claims 1-24, wherein the polymer represents a single crystal asymmetric unit.

26. The computer system of any one of claims 1-24, wherein the plurality of residues includes one or more second residues that are crystallographic symmetry mates of one or more first residues in the plurality of residues and the graph includes a definition of the default asymmetric unit of the polymer.

27. The computer system of any one of claims 1-26, wherein each residue in the plurality of residues is one of twenty naturally occurring amino acids.

28. The computer system of any one of claims 1-27, wherein each node in the plurality of nodes represents an atom as an encoded tuple that includes an encoding of residue type of the residue the atom is in the name of the atom in the residue.

29. The computer system of any one of claims 1-28, wherein the at least one program further comprises instructions for training the first trained partial-context graph neural network, the second trained partial-context graph neural network, and each trained full-context graph neural network in the plurality of trained full-context graph neural networks using a loss function that trains unambiguous side chain dihedral angles as a regression task and ambiguous side chain dihedral angles by considering the lower of the two possible losses attributable to the ambiguous side chain dihedral angle Xᵢ.

30. The computer system of claim 29, wherein the regression task a mean squared error loss function, a mean absolute error loss function, a Huber loss function, a Log-Cosh loss function, or a quantile loss function.

31. The computer system of claim 30, wherein a first loss in the two possible losses is for a side chain dihedral angle value for Xᵢ and the second loss in the two possible losses is a for a side chain dihedral angle value for Xᵢ−π.

32. The computer system of any one of claims 1-30, wherein the at least one program further comprises instructions for using the elucidated side chain dihedral angle values for the plurality of residues to determine an interaction score between the polymer and a composition.

33. The computer system of claim 32, wherein
the polymer is an enzyme,
the composition is being screened in silico to assess an ability to inhibit an activity of the enzyme, and
the interaction score is a calculated binding coefficient of the composition to the first enzyme.

34. The computer system of claim 33, wherein the composition has a molecular weight of 2000 Daltons or less.

35. The computer system of claim 33, wherein the composition satisfies any two or more rules, any three or more rules, or all four rules of the Lipinski's rule of Five: (i) not more than five hydrogen bond donors, (ii) not more than ten hydrogen bond acceptors, (iii) a molecular weight under 500 Daltons, and (iv) a Log P under 5.

36. The computer system of claim 32, wherein
the polymer is a first protein,
the composition is a second protein being screened in silico to assess an ability to bind to the first protein in order to inhibit or enhance an activity of the first protein, and
the interaction score is a calculated binding coefficient of the second protein to the first protein.

37. The computer system of claim 32, wherein
the polymer is a first Fc fragment of a first type,
the composition is a second protein is Fc fragment of a second type, and
the interaction score is a calculated binding coefficient of the second Fc fragment to the first Fc fragment.

38. The computer system of any one of claims 32-37, wherein the at least one program further comprises instructions for using the interaction score of the composition to develop a treatment of a medical condition associated with the polymer.

39. The computer system of claim 38, wherein the treatment comprises the composition and one or more excipient and/or one or more pharmaceutically acceptable carrier and/or one or more diluent.

40. The computer system of claim 38 or 39, wherein the medical condition is inflammation or pain.

41. The computer system of claim 38 or 39, wherein the medical condition is a disease.

42. The computer system of claim 38 or 39, wherein the medical condition is asthma, an autoimmune disease, autoimmune lymphoproliferative syndrome (ALPS), cholera, a viral infection, Dengue fever, an E. coli infection, Eczema, hepatitis, Leprosy, Lyme Disease, Malaria, Monkeypox, Pertussis, a Yersinia pestis infection, primary immune deficiency disease, prion disease, a respiratory syncytial virus infection, Schistosomiasis, gonorrhea, genital herpes, a human papillomavirus infection, chlamydia, syphilis, Shigellosis, Smallpox, STAT3 dominant-negative disease, tuberculosis, a West Nile viral infection, or a Zika viral infection.

43. The computer system of any one of claims 38-42, wherein the at least one program further comprises instructions for providing instructions to a medical practitioner to provide the treatment of the medical condition to a subject in need of treatment of the medical condition.

44. The computer system of any one of claims 1-31, wherein the polymer is a protein with one or more mutations introduced into the protein and the at least one program further comprises instructions for using the elucidated side chain dihedral angle values for the plurality of residues to determine an effect of the one or more mutations on an activity of the protein relative to an activity of a wild-type naturally occurring version of the protein.

45. The computer system of any one of claims 1-44, wherein the plurality of residues comprises each residue of the polymer.

46. A non-transitory computer readable storage medium storing one or more computational modules for molecular modeling, the one or more computational modules collectively comprising instructions for:
(A) obtaining a graph of at least a portion of a polymer, wherein the graph comprises a plurality of nodes and a plurality of edges, each node in the plurality of nodes representing a main chain atom of the polymer, and each respective edge in the plurality of edges encodes at least (i) a corresponding distance relationship between a corresponding pair of nodes in the plurality of nodes and (ii) a binary indicator that indicates whether or not the corresponding pair of nodes represents a pair of atoms covalently bound to each other in the polymer, and wherein the portion of a polymer comprises a plurality of residues, at least two of which have one or more side chain dihedral angles in a set of side chain dihedral angles;
(B) sequentially inputting each first partial-context subgraph in a plurality of first partial-context subgraphs of the graph into a first trained partial-context graph neural network having at least 500 parameters, thereby obtaining a plurality of first instances of calculated first side chain dihedral angles for the plurality of residues;
(C) updating the graph up to the first side chain dihedral angle of each residue in the plurality of residues using the plurality of first instances of calculated first side chain dihedral angles;
(D) sequentially inputting each second partial-context subgraph in a plurality of second partial-context subgraphs of the graph into a second trained partial-context graph neural network having at least 500 parameters, thereby obtaining a plurality of first instances of calculated second side chain dihedral angles for residues in the plurality of residues;
(E) updating the graph up to a level of a second side chain dihedral angle using the plurality of first instances of calculated second side chain dihedral angles; and
(F) updating the graph with updated side chain dihedral angle values obtained by sequentially inputting a plurality of full-context subgraphs, each full-context subgraph in the plurality of full-context subgraphs associated with a different residue in the plurality of residues, into a plurality of trained full-context graph neural networks, each having at least 500 parameters, thereby elucidating the side chain dihedral angle values for the plurality of residues.

47. A method of for molecular modeling, the method comprising:
at a computer system comprising a memory:
(A) obtaining a graph of at least a portion of a polymer, wherein the graph comprises a plurality of nodes and a plurality of edges, each node in the plurality of nodes representing a main chain atom of the polymer, and each respective edge in the plurality of edges encodes at least (i) a corresponding distance relationship between a corresponding pair of nodes in the plurality of nodes and (ii) a binary indicator that indicates whether or not the corresponding pair of nodes represents a pair of atoms covalently bound to each other in the polymer, and wherein the portion of a polymer comprises a plurality of residues, at least two of which have one or more side chain dihedral angles in a set of side chain dihedral angles;
(B) sequentially inputting each first partial-context subgraph in a plurality of first partial-context subgraphs of the graph into a first trained partial-context graph neural network having at least 500 parameters, thereby obtaining a plurality of first instances of calculated first side chain dihedral angles for the plurality of residues;
(C) updating the graph up to the first side chain dihedral angle of each residue in the plurality of residues using the plurality of first instances of calculated first side chain dihedral angles;
(D) sequentially inputting each second partial-context subgraph in a plurality of second partial-context subgraphs of the graph into a second trained partial-context graph neural network having at least 500 parameters, thereby obtaining a plurality of first instances of calculated second side chain dihedral angles for residues in the plurality of residues;
(E) updating the graph up to a level of a second side chain dihedral angle using the plurality of first instances of calculated second side chain dihedral angles; and
(F) updating the graph with updated side chain dihedral angle values obtained by sequentially inputting a plurality of full-context subgraphs, each full-context subgraph in the plurality of full-context subgraphs associated with a different residue in the plurality of residues, into a plurality of trained full-context graph neural networks, each having at least 500 parameters, thereby elucidating the side chain dihedral angle values for the plurality of residues.

48. The method of claim 47, wherein the sequentially inputting (B) comprises, for each respective residue in the plurality of residues, inputting a corresponding first partial-context subgraph, in the plurality of first partial-context subgraphs of the graph, drawn from the nodes in the graph that represent atoms of the respective residue or atoms of the polymer proximate to the respective residue, into the first trained partial-context graph neural network, thereby obtaining a first instance of a corresponding calculated first side chain dihedral angle for the respective residue.

49. The method of claim 48, wherein the updating (C) comprises, for each respective residue in the plurality of residues, using the corresponding first instance of the corresponding calculated first side chain dihedral angle to update the graph of the polymer to include nodes and edges for atoms of the respective residue up to the first side chain dihedral angle of the respective residue.

50. The method of any one of claims 47-49, wherein the sequentially inputting (D) comprises, for each respective residue in the plurality of residues having a second side chain dihedral angle, inputting a corresponding second partial-context subgraph, in the plurality of second partial-context subgraphs of the graph, drawn from the nodes in the graph that represent backbone atoms or side chain atoms of up to the first side chain dihedral angle of (i) the respective residue or (ii) residues proximate to the respective residue, into the second trained partial-context graph neural network, thereby obtaining a first instance of a corresponding calculated second side chain dihedral angle for the respective residue.

51. The method of claim 50, wherein the updating (E) comprises, for each respective residue in the plurality of residues having a second side chain dihedral angle, using the corresponding first instance of the corresponding calculated second side chain dihedral angle to update the graph to include nodes and edges for atoms of the respective residue up to the second dihedral angle.

52. The method of any one of claims 47-51, wherein the updating (F) comprises:
(i) for each respective residue in the plurality of residues, inputting a corresponding first full-context subgraph drawn from the nodes in the graph, other than side chain atoms beyond the C_{β} carbon of the respective residue, into a first trained full-context graph neural network in the plurality of trained full-context graph neural networks, thereby obtaining a second instance of a corresponding calculated first side chain dihedral angle for the respective residue,
(ii) for each respective residue in the plurality of residues, using the second instance of the corresponding calculated first side chain dihedral angle to update the corresponding distance relationship of edges in the graph affected by the second instance of the corresponding calculated first side chain dihedral angle,
(iii) for each respective residue in the plurality of residues having a second side chain dihedral angle, inputting a corresponding second full-context subgraph drawn from the nodes in the graph, other than side chain atoms of the respective residue beyond the first dihedral angle, into a second trained full-context graph neural network in the plurality of trained full-context graph neural networks, thereby obtaining a second instance of a corresponding calculated second side chain dihedral angle for the respective residue, and
(iv) for each respective residue in the plurality of residues having a second side chain dihedral angle, using the second instance of the corresponding calculated second side chain dihedral angle to update the distance relationship of each edge in the graph affected by the second instance of the corresponding calculated second side chain dihedral angle.

53. The method of any one of claims 47-52, wherein the corresponding distance relationship between a corresponding pair of nodes i and j in the plurality of nodes is of the form e^{-r}^{ᵢⱼ}^{²}^{/κ}, wherein
rᵢⱼ is a distance between three-dimensional coordinates for node i and three-dimensional coordinates for node j, and
κ is a square of a cuttoff distance.

54. The method of claim 53, wherein rᵢⱼ is in units of Å and κ is 100 Å².

55. The method of any one of claims 47-54, wherein each respective edge in the plurality of edges further encodes a directional feature between a corresponding pair of nodes.

56. The method of claim 55, wherein each respective node in the corresponding pair of nodes i, j is assigned its own local three-dimensional reference frame and the directional feature is encoded as a 1×3 additional features representing a projection of the three dimensional coordinates of the node i onto to the local three-dimensional reference frame of node j in an edge eᵢⱼ, and 1×3 additional features representing a projection of the three dimensional coordinates of the node j onto to the local three-dimensional reference frame of the node i in an edge eⱼᵢ.

57. The method of any one of claims 47-56, wherein the polymer is a polypeptide.

58. The method of any one of claims 47-56, wherein the polymer is an antigen-antibody complex.

59. The method of any one of claims 47-58, wherein the plurality of residues comprises 50 or more residues.

60. The method of claim 48, wherein the first instance of the corresponding calculated first side chain dihedral angle for the respective residue is the X₁ side chain dihedral angle for the respective residue.

61. The method of claim 60, wherein the first instance of the corresponding calculated second side chain dihedral angle for the respective residue is the X₂ side chain dihedral angle for the respective residue.

62. The method of claim 61, further comprising;
prior to the updating (F), for each respective residue in the plurality of residues having a X₃ dihedral angle, inputting a corresponding third partial-context subgraph drawn from the nodes in the graph that represent backbone atoms or side chain atoms of up to the second side chain dihedral angle of (i) the respective residue or (ii) residues proximate to the respective residue, into a third trained partial-context graph neural network having at least 500 parameters, thereby obtaining a first instance of a corresponding calculated X₃ dihedral angle for the respective residue,
for each respective residue in the plurality of residues having a X₃ dihedral angle, using the corresponding first instance of the corresponding calculated X₃ dihedral angle to update the graph to include nodes and edges for atoms of the respective residue up to the X₃ dihedral angle, and
the updating (F) further comprises:
(v) for each respective residue in the plurality of residues having a X₃ dihedral angle, inputting a corresponding third full-context subgraph drawn from the nodes in the graph, other than side chain atoms of the respective residue beyond the second dihedral angle, into a third trained full-context graph neural network in the plurality of trained full-context graph neural networks, thereby obtaining a second instance of a corresponding calculated X₃ dihedral angle for the respective residue, and
(vi) for each respective residue in the plurality of residues having a X₃ dihedral angle, using the second instance of the corresponding calculated X₃ dihedral angle to update the distance relationship of each edge in the graph affected by the second instance of the corresponding calculated X₃ dihedral angle.

63. The method of claim 61, the method further comprising:
prior to the updating (F), for each respective residue in the plurality of residues having a X₄ dihedral angle, inputting a corresponding fourth partial-context subgraph drawn from the nodes in the graph that represent backbone atoms or side chain atoms of up to the X₃ dihedral angle of (i) the respective residue or (ii) residues proximate to the respective residue, into a fourth trained partial-context graph neural network having at least 500 parameters, thereby obtaining a first instance of a corresponding calculated X₄ dihedral angle for the respective residue,
for each respective residue in the plurality of residues having a X₄ dihedral angle, using the corresponding first instance of the corresponding calculated X₄ dihedral angle to update the graph to include nodes and edges for atoms of the respective residue through the X₄ dihedral angle, and
the updating (F) further comprises:
(vi) for each respective residue in the plurality of residues having a X₄ dihedral angle, inputting a corresponding fourth full-context subgraph drawn from the nodes in the graph, other than side chain atoms of the respective residue beyond the X₃ dihedral angle, into a fourth trained full-context graph neural network in the plurality of trained full-context graph neural networks, thereby obtaining a second instance of a corresponding calculated X₄ dihedral angle for the respective residue, and
(vi) for each respective residue in the plurality of residues having a X₄ dihedral angle, using the second instance of the corresponding calculated X₄ dihedral angle to update the distance relationship of each edge in the graph affected by the second instance of the corresponding calculated X₄ dihedral angle.

64. The method of claim 48, wherein the backbone atoms of the polymer proximate to the respective residue are a cutoff number of atoms in the polymer that are closest to the respective residue.

65. The method of claim 64, wherein the cutoff number of atoms is between 20 and 80 atoms.

66. The method of any one of claims 47-65, wherein the first trained partial-context graph neural network, the second trained partial-context graph neural network, and each trained full-context graph neural network in the plurality of trained full-context graph neural networks is a message passing graph neural network.

67. The method of claim 66, wherein the first trained partial-context graph neural network, the second trained partial-context graph neural network, and each trained full-context graph neural network in the plurality of trained full-context graph neural networks comprises an embedding layer for receiving embedded graph information associated with a residue in the polymer, followed by a plurality of layers that each convolve over both a plurality of edge attributes and a plurality of node attributes, followed by an average pooling layer employed to the nodes corresponding to atoms in the respective residue, followed by a multi-layered perceptron with an activation function having two output channels, wherein the output channels give a sine and a cosine value for a side chain dihedral angle of the respective residue.

68. The method of claim 67, wherein the activation function is tanh.

69. The method of any one of claims 47 through 68, wherein the at least one program further comprises instructions for:
repeating the sequentially inputting (B), updating (C), sequentially inputting (D), updating (E), and updating (F) until a side chain dihedral angle convergence criterion is satisfied.

70. The method of claim 69, wherein the side chain dihedral angle convergence criterion is an average change in side chain dihedral angle across the plurality of residues after repetition of the sequentially inputting (B), updating (C), sequentially inputting (D), updating (E), and updating (F) dropping below a threshold value.

71. The method of any one of claims 47-70, wherein the polymer represents a single crystal asymmetric unit.

72. The method of any one of claims 47-71, wherein the plurality of residues includes one or more second residues that are crystallographic symmetry mates of one or more first residues in the plurality of residues and the graph includes a definition of the default asymmetric unit of the polymer.

73. The method of any one of claims 47-72, wherein each residue in the plurality of residues is one of twenty naturally occurring amino acids.

74. The method of any one of claims 47-73, wherein each node in the plurality of nodes represents an atom as an encoded tuple that includes an encoding of residue type of the residue the atom is in the name of the atom in the residue.

75. The method of any one of claims 47-74, wherein the at least one program further comprises instructions for training the first trained partial-context graph neural network, the second trained partial-context graph neural network, and each trained full-context graph neural network in the plurality of trained full-context graph neural networks using a loss function that trains unambiguous side chain dihedral angles as a regression task and ambiguous side chain dihedral angles by considering the lower of the two possible losses attributable to the ambiguous side chain dihedral angle Xᵢ.

76. The method of claim 75, wherein the regression task a mean squared error loss function, a mean absolute error loss function, a Huber loss function, a Log-Cosh loss function, or a quantile loss function.

77. The method of claim 76, wherein a first loss in the two possible losses is for a side chain dihedral angle value for Xᵢ and the second loss in the two possible losses is a for a side chain dihedral angle value for Xᵢ−π.

78. The method of any one of claims 47-77, wherein the at least one program further comprises instructions for using the elucidated side chain dihedral angle values for the plurality of residues to determine an interaction score between the polymer and a composition.

79. The method of claim 78, wherein
the polymer is an enzyme,
the composition is being screened in silico to assess an ability to inhibit an activity of the enzyme, and
the interaction score is a calculated binding coefficient of the composition to the first enzyme.

80. The method of claim 78 or 79, wherein the composition has a molecular weight of 2000 Daltons or less.

81. The method of any one of claims 78-80, wherein the composition satisfies any two or more rules, any three or more rules, or all four rules of the Lipinski's rule of Five: (i) not more than five hydrogen bond donors, (ii) not more than ten hydrogen bond acceptors, (iii) a molecular weight under 500 Daltons, and (iv) a Log P under 5.

82. The method of claim 78, wherein
the polymer is a first protein,
the composition is a second protein being screened in silico to assess an ability to bind to the first protein in order to inhibit or enhance an activity of the first protein, and
the interaction score is a calculated binding coefficient of the second protein to the first protein.

83. The method of claim 78, wherein
the polymer is a first Fc fragment of a first type,
the composition is a second protein is Fc fragment of a second type, and
the interaction score is a calculated binding coefficient of the second Fc fragment to the first Fc fragment.

84. The method of any one of claims 78-83, wherein the at least one program further comprises instructions for using the interaction score of the composition to develop a treatment of a medical condition associated with the polymer.

85. The method of claim 84, wherein the treatment comprises the composition and one or more excipient and/or one or more pharmaceutically acceptable carrier and/or one or more diluent.

86. The method of claim 84 or 85, wherein the medical condition is inflammation or pain.

87. The method of claim 84 or 85, wherein the medical condition is a disease.

88. The method of claim 84 or 85, wherein the medical condition is asthma, an autoimmune disease, autoimmune lymphoproliferative syndrome (ALPS), cholera, a viral infection, Dengue fever, an E. coli infection, Eczema, hepatitis, Leprosy, Lyme Disease, Malaria, Monkeypox, Pertussis, a Yersinia pestis infection, primary immune deficiency disease, prion disease, a respiratory syncytial virus infection, Schistosomiasis, gonorrhea, genital herpes, a human papillomavirus infection, chlamydia, syphilis, Shigellosis, Smallpox, STAT3 dominant-negative disease, tuberculosis, a West Nile viral infection, or a Zika viral infection.

89. The method of any one of claims 84-88, wherein the method further comprises treating the medical condition by administering the treatment to a subject in need of treatment of the medical condition.

90. The method of any one of claims 47-89, wherein the polymer is a protein with one or more mutations introduced into the protein and the at least one program further comprises instructions for using the elucidated side chain dihedral angle values for the plurality of residues to determine an effect of the one or more mutations on an activity of the protein relative to an activity of a wild-type naturally occurring version of the protein.

91. The method of any one of claims 47-90, wherein the plurality of residues comprises each residue of the polymer.
